(19) United States
(12) Patent Application Publication
(10) Pub. No.: US 2025/0161279 A1
(43) Pub. Date: May 22, 2025

(54) METHODS OF TREATING MULTIPLE MYELOMA USING BCL-2 INHIBITOR

(71) Applicants: BeiGene Switzerland GmbH, Basel (CH); Onyx Pharmaceuticals, Inc., Thousand Oaks, CA (US)

(72) Inventors: Shuran LI, Beijing (CN); Rocco CRESCENZO, San Mateo, CA (US); Nan HU, Beijing (CN); Xiaomin SONG, Beijing (CN)

(73) Assignees: BeiGene Switzerland GmbH, Basel (CH); Onyx Pharmaceuticals, Inc., Thousand Oaks, CA (US)

(21) Appl. No.: 19/030,242

(22) Filed: Jan. 17, 2025

Related U.S. Application Data
(63) Continuation of application No. PCT/CN2023/108470, filed on Jul. 20, 2023.
(60) Provisional application No. 63/369,000, filed on Jul. 21, 2022.

(51) Int. Cl. A61K 31/437; A61K 31/5377; A61K 31/573; A61P 35/00

(52) U.S. Cl. CPC A61K 31/437; A61K 31/5377; A61K 31/573; A61P 35/00

(57) ABSTRACT

The present disclosure provides methods of treating multiple myeloma in a subject with a Bcl-2 inhibitor, in particularly 2-((1H-pyrrolo [2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof, or in combination with dexamethasone.

### CROSS-REFERENCE TO RELATED APPLICATIONS

This application is a continuation of International Application No. PCT/CN2023/108470, filed on Jul. 20, 2023, which claims the benefit of U.S. Provisional Application No. 63/369,000, filed on Jul. 21, 2022, the disclosures of which are hereby incorporated by reference in their entireties.

### FIELD OF THE DISCLOSURE

Disclosed herein are methods of treating multiple myeloma with a Bcl-2 inhibitor, in particularly 2-((1H-pyrrolo [2,3-b]pyridin-5-yl) oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro [3.5]nonan-7-yl)benzamide or a pharmaceutically acceptable salt thereof, or in combination with dexamethasone.

### BACKGROUND OF THE DISCLOSURE

Multiple myeloma (MM) is a malignancy involving terminally differentiated plasma cells that accumulate in the bone marrow, leading to bone destruction and marrow failure. Myeloma represents the classic multistep transformation process with an initial premalignant stage, monoclonal gammopathy of unknown significance (MGUS), demonstrating a number of recurrent cytogenetic abnormalities as well as gene expression changes. The most common cytogenetic abnormalities include hyperdiploidy, del (13), hypodiploid, t (11;14), pseudo-diploid, t (4;14), and del (17). (Sawyer J R. The prognostic significance of cytogenetics and molecular profiling in multiple myeloma. Cancer Genetics. 2011;204 (1): P3-12). The molecular subgroup of t (11;14) MM is associated with high levels of B-cell lymphoma-2 (Bcl-2) and low myeloid leukemia cell differentiation protein (MCL-1)/Bcl-extra large (XL) expression (Touzeau C, Maciag P, Amiot M, et al. Targeting Bcl-2 for the treatment of multiple myeloma. Leukemia. 2018;32(9):1899-1907.).

The Bcl-2 family consists of numerous pro-and anti-apoptotic proteins. The anti-apoptotic proteins include Bcl-2, Bcl-XL, MCL-1, Bcl-W, and Bcl-2-related protein A1 (Bfl-1). Specifically, the pro-apoptotic proteins bind via their Bcl-2 Homology 3 (BH3) domain to a hydrophobic groove in the anti-apoptotic proteins which in turn neutralizes the activator BH3 pro-apoptotic proteins, thereby shifting the balance of anti-and pro-apoptotic proteins in favor of anti-apoptosis and providing MM cells with a survival advantage (Gong J-N, Khong T, Segal D, et al. Hierarchy for targeting pro-survival BCL2 family proteins in multiple myeloma: pivotal role of MCL1. Blood. 2016;128(14):1834-44.; Gupta V A, Ackley J, Kaufman J L, et al. BCL2 family inhibitors in the biology and treatment of multiple myeloma. Blood Lymph Cancer: Targets Ther. 2021;11:11-24).

Venetoclax (ABT-199) is a Bcl-2 inhibitor approved for treating patients with chronic lymphocytic leukemia (CLL) and acute myeloid leukemia (AML). It is also the first in class orally bioavailable Bcl-2 selective BH3 mimetic, demonstrated strong clinical activity against MM cell lines alone or in combination with other agents. An initial Phase 1 study in patients with relapsed/refractory (R/R) MM treated with venetoclax monotherapy demonstrated an overall response rate (ORR) of 21% (14 of 66 patients); 15% (10 of 66 patients) achieved very good partial response (VGPR) or better. In a subgroup analysis of the t (11;14) patients, the ORR was 40% (Kumar S, Kaufman J L, Gasparetto C, et al. Efficacy of venetoclax as targeted therapy for relapsed/refractory t (11;14) multiple myeloma. Blood. 2017;130(22):2401-09). The addition of venetoclax to bortezomib and dexamethasone significantly improved the response rate and progression-free survival (PFS); however, overall survival (OS) favored the placebo group, indicating an unfavorable risk-benefit ratio in unselected patients with multiple myeloma (Kumar S, Harrison S J, Cavo M, et al. Venetoclax or placebo in combination with bortezomib and dexamethasone in patients with relapsed or refractory multiple myeloma (BELLINI): a randomized, double-blinded, multicentre, phase 3 trail. Lancet Oncol. 2020b;21(12):1630-42.). At the American Society of Clinical Oncology 2020 Meeting, updated results of the BELLINI trial were presented and showed a median OS in all patients of 33.5 months versus not reached (hazard ratio [HR]=1.46 (0.91, 2.34) in the venetoclax and placebo arms, respectively. The median overall survival in the t (11;14) or BCL-2 high subpopulation was not reached in either arm with a HR=0.97 (0.43, 2.17) (Kumar S, Harrison S J, Cavo M, et al. Updated results from BELLINI, a phase III study of venetoclax or placebo in combination with bortezomib and dexamethasone in relapsed/refractory multiple myeloma. J Clin Oncol. 2020a; 38 (15_suppl): 8509). These findings have led the Food and Drug Administration (FDA) to issue a safety warning. The interim trial results demonstrated an increased risk of death for patients receiving venetoclax compared to the control group. The Phase 3 trial compared venetoclax/dexamethasone/bortezomib to placebo/dexamethasone/bortezomib and showed a median progression free survival (PFS) of 22.4 versus 11.5 months (p=0.010; hazard ratio [HR]=0.630; 95% CI: 0.443 to 0.897) (Multiple Myeloma Hub. EHA 2019 | Results of the phase III BELLINI trial: VenBd for RRMM. https://multiplemyelomahub.com/medical-information/eha-2019-or-results-of-the-phase-iiibellini-trial-venbd-for-rrmm.).

WO2019/210828A disclosed a series of compounds having the following Formulas (III-B), (III-C), (III-D) or (III-E), or a stereoisomer thereof, or a pharmaceutically acceptable salt thereof, as Bcl-2 inhibitors. The compounds disclosed in WO2019/210828A are potent and selective Bcl-2 protein inhibitors.

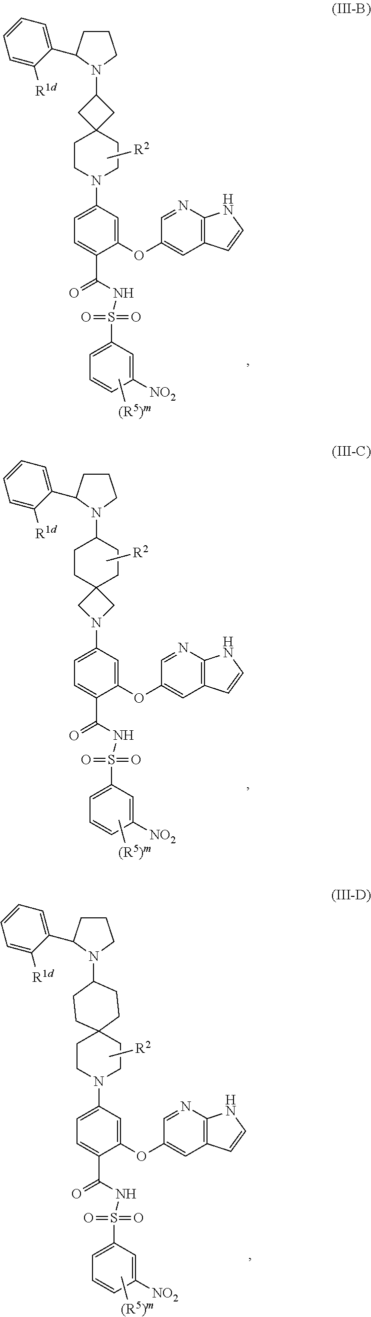

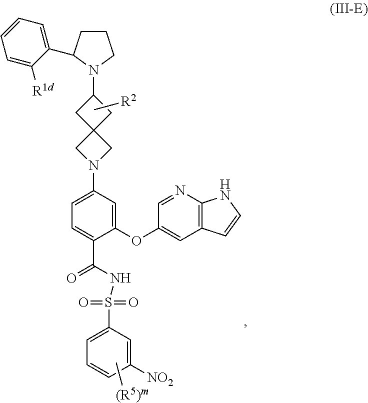

### SUMMARY OF THE DISCLOSURE

The inventors of the present disclosure have found that a Bcl2 inhibitor having Formulas (III-B), (III-C), (III-D) or (III-E), in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) or a pharmaceutically acceptable salt thereof, in combination with dexamethasone produced significant inhibition of tumor growth in multiple myeloma (MM) as compared with the efficacy of each therapeutic as a single agent. Further, the combination therapy demonstrated potential safety and efficacy.

In a first aspect, disclosed herein is a method of treating multiple myeloma (MM) with a Bcl-2 inhibitor, wherein the Bcl-2 inhibitor is a compound represented by the following Formulas (III-B), (III-C), (III-D) or (III-E),

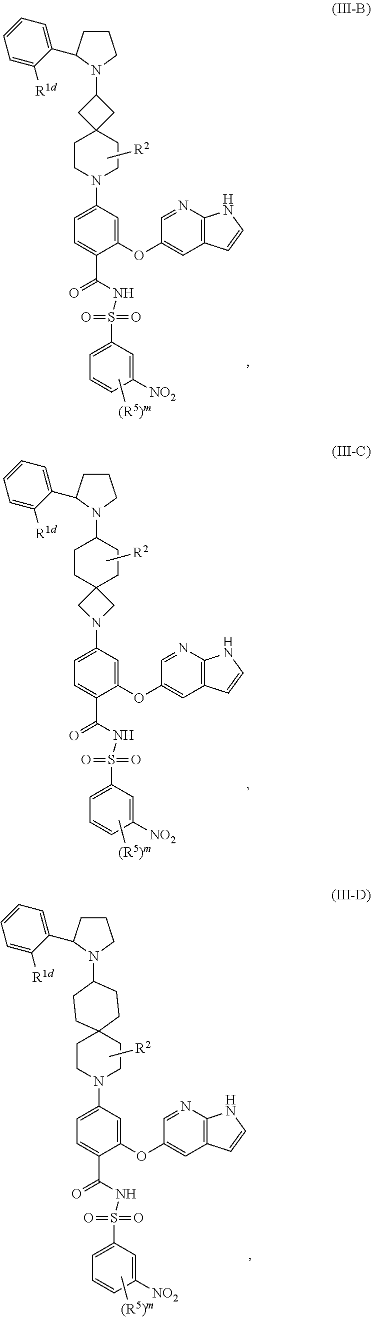

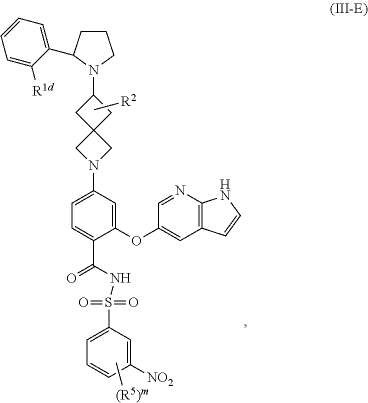

or a pharmaceutically acceptable salt thereof, or a stereoisomer thereof,
wherein,
R², at each occurrence, is independently selected from the group consisting of hydrogen, halogen, or -C₁₋₈alkyl optionally substituted with halogen;
R^{1d}, at each occurrence, is independently halogen, -C₁₋₈alkyl, -C₂₋₈alkenyl, -C₂₋₈alkynyl, cycloalkyl, heterocyclyl, aryl, heteroaryl, oxo, —CN, —NO₂, —OR^{Ba}, —SO₂R^{Ba}, —COR^{Ba}, —CO₂R^{Ba}, —CONR^{Ba}R^{Bb}, —C(═NR^{Ba})NR^{Bb}R^{Bc}, —NR^{Ba}R^{Bb}, —NR^{Ba}COR^{Bb}, —NR^{Ba}CONR^{Bb}R^{Bc}, —NR^{Ba}CO₂R^{Bb}, —NR^{Ba}SONR^{Bb}R^{Bc}, —NR^{Ba}SO₂NR^{Bb}R^{Be}, or —NR^{Ba}SO₂R^{Bb}; wherein said -C₁₋₈alkyl, -C₂₋₈alkenyl, -C₂₋₈alkynyl, cycloalkyl, heterocyclyl, aryl or heteroaryl are each independently optionally substituted with 1 to 4 substituents R^{Bd};
R^{Ba}, R^{Bb}, and R^{Be}, are each independently hydrogen, -C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, -C₂₋₈alkynyl, cycloalkyl, heterocyclyl, aryl, or heteroaryl is optionally substituted with halogen, hydroxy, —NH₂ or —N(C₁₋₆alkyl)₂, -C₁₋₈alkyoxy, cycloalkyl, heterocyclyl, aryl, or heteroaryl;
R^{Bd}, at each occurrence, is independently hydrogen, halogen, oxo, —CN, —NO₂, -C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, -C₂₋₈alkynyl, cycloalkyl, heterocyclyl, aryl, or heteroaryl is optionally substituted with halogen, hydroxy, -C₁₋₈alkyoxy, cycloalkyl, heterocyclyl, aryl, or heteroaryl;
m is an integer of 1-4;
R⁵ is -L⁵-CyC,
   Wherein L⁵ is a direct bond, -(CR^{a}R^{b})ₜ₋₁-, -(CR^{a}R^{b})ₜ₋₁-(CR^{c}═CR^{d})-(CR^{a}R^{b})ᵥ₋₁-, -(CR^{a}R^{b})ₜ₋₁-(C≡C)-(CR^{a}R^{b})ᵥ₋₁-, —O—, —S—, —S(O)—, —SO₂-, —C(O)—, C(O)O—, —OC(O)—, —NR^{a}-, —C(O)NR^{a}-, —NR^{a}C(O)—, —NR^{a}C(O)O—, —NR^{a}C(O)NR^{b}-, —SO₂NR^{a}-, —NR^{a}SO₂-, —NR^{a}S(O)₂NR^{b}-, -NR^{a}S(O)NR^{b}-, —C(O)NR^{a}SO₂-, —C(O)NR^{a}SO—, or —C(═NR^{a})NR^{b}-, wherein t and v, at each occurrence, are independently a number of 1 to 7, and one or two CR^{a}R^{b} moieties in -(CR^{a}R^{b})-, -(CR^{a}R^{b})ₜ₋₁-(CR^{c}═CR^{d})-(CR^{a}R^{b})ᵥ₋₁-, —(CR^{a}R^{b})ₜ₋₁-(C≡C)-(CR^{a}R^{b})ᵥ₋₁- are un-replaced or replaced with one or more moieties selected from O, S, SO, SO₂, C(O) and NR^{a};
   CyC is cycloalkyl, heterocyclyl, aryl, or heteroaryl, each of which is optionally substituted with one or two substituents R^{5a};
   R^{5a}, at each occurrence, is independently selected from hydrogen, halogen, cyano, oxo, —NO₂, —OR^{5b}, —SR^{5b}, —NR^{5b}R^{5c}, —COR^{5b}, —SO₂R^{5b}, —C(═O)OR^{5b}, —C(═O)NR^{5b}R^{5c}, —C(═NR^{5b})NR^{5c}R^{5d}, —N(R^{5b})C(═O)R^{5c}, —N(R^{5b})C(═O)OR^{5c}, —N(R^{5b})C(O)NR^{5c}R^{5d}, —N(R^{5b})S(O)NR^{5c}R^{5d}, —N(R^{5b})S(O)₂NR^{5c}R^{5d}, —NR^{5b}SO₂R^{5c}, -C₁₋₈alkyl, -C₂₋₈alkenyl, -C₂₋₈alkynyl, -cycloalkyl, heterocyclyl, aryl, or heteroaryl each of said -C₁₋₈alkyl, -C₂₋₈alkenyl, -C₂₋₈alkynyl, -cycloalkyl, heterocyclyl, aryl, or heteroaryl is optionally substituted with one or two substituents R^{5e};
      wherein R^{5b}, R^{5c}, and R^{5d} are each independently hydrogen, -C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, C₂₋₈alkynyl, -cycloalkyl, heterocyclyl, aryl, or heteroaryl is optionally substituted with one or two substituents R^{5e};
   R^{5e}, at each occurrence, is independently selected from hydrogen, halogen, cyano, oxo, —NO₂, —OR^{5f}, —SR^{5f}, —NR^{5f}R^{5g}, —COR^{5f}, —SO₂R^{5f}, —C(═O)OR^{5f}, —C(═O)NR^{5f}R^{5g}, —C(═NR^{5f})NR^{5g}R^{5h}, —N(R^{5f})C(═O)R^{5g}, —N(R^{5f})C(═O)OR^{5g}, —N(R^{5f})C(O)NR^{5g}R^{5h}, —N(R^{5f})S(O)NR^{5g}R^{5h}, —N(R^{5f})S(O)₂NR^{5g}R^{5h}, —NR^{5f}SO₂R^{5g}, -C₁₋₈alkyl, -C₂₋₈alkenyl, -C₂₋₈alkynyl, -cycloalkyl, heterocyclyl, aryl, or heteroaryl;
      R^{5f}, R^{5g}, and R^{5h} are each independently hydrogen, -C₁₋₈alkyl, -C₂₋₈alkenyl, -C₂₋₈alkynyl, cycloalkyl, heterocyclyl, aryl, or heteroaryl;
or, two adjacent R⁵ on the phenyl ring together with the phenyl ring form a benzo ring, said ring is optionally substituted with halogen, oxo, cyano, —NO₂, —OR⁵ⁱ, —SR⁵ⁱ, —NR⁵ⁱR^{5j}, —COR⁵ⁱ, —SO₂R⁵ⁱ, —C(═O)OR⁵ⁱ, —C(═O)NR⁵ⁱR^{5j}, —C(═NR⁵ⁱ)NR^{5j}R^{5k}, —N(R^{5l})C(═O)R^{5j}, —N(R⁵ⁱ)C(═O)OR^{5j}, —N(R⁵ⁱ)C(O)NR^{5j}R^{5k}, —N(R⁵ⁱ)S(O)NR^{5j}R^{5k}, —N(R⁵ⁱ)S(O)₂NR⁵ⁱR^{5k}, —NR⁵ⁱSO₂R^{5k}, -C₁₋₈alkyl, -C₂₋₈alkenyl, -C₂₋₈alkynyl, -cycloalkyl, heterocyclyl, aryl, or heteroaryl;
   R⁵ⁱ, R^{5j}, and R^{5k} are independently hydrogen, -C₁₋₈alkyl, -C₂₋₈alkenyl, -C₂₋₈alkynyl, cycloalkyl, heterocyclyl, aryl, or heteroaryl, each of said-C₁₋₈alkyl, -C₂₋₈alkenyl, -C₂₋₈alkynyl, cycloalkyl, heterocyclyl, aryl, or heteroaryl is optionally substituted with halogen, hydroxy or -C₁₋₈alkyoxy;
R^{a}, R^{b}, R^{c}, and R^{d} at each occurrence, are independently hydrogen, -C₁₋₈alkyl, -C₂₋₈alkenyl, -C₂₋₈alkynyl, cycloalkyl, heterocyclyl, aryl, or heteroaryl, said-C₁₋₈alkyl, -C₂₋₈alkenyl, -C₂₋₈alkynyl, cycloalkyl, heterocyclyl, aryl, or heteroaryl are each independently substituted with-CN, halogen, —NO₂, —NR^{e}R^{f}, oxo, —OR^{e}, or —SR^{e}; and
   wherein R^{e} and R^{f} are each independently hydrogen, C₁₋₈alkyl, C₁₋₈alkoxy-C₁₋₈alkyl-, C₂₋₈alkenyl, C₂₋₈alkynyl, cycloalkyl, aryl, heterocyclyl, or heteroaryl.

In a second aspect, disclosed herein is a method of treating multiple myeloma (MM) with a Bcl-2 inhibitor, wherein the Bcl-2 inhibitor is a compound represented by the following Formulas (III-B), (III-C), (III-D) or (III-E), or a stereoisomer thereof, or a pharmaceutically acceptable salt thereof, in combination with dexamethasone.

In a third aspect, disclosed herein is a method of treating multiple myeloma (MM) with a Bcl-2 inhibitor, wherein the Bcl-2 inhibitor is a compound represented by the following Formulas (III-B), (III-C), (III-D) or (III-E), or a stereoisomer thereof, or a pharmaceutically acceptable salt thereof, in combination with a proteasome inhibitor and dexamethasone.

In a fourth aspect, disclosed herein is a Bcl-2 inhibitor of Formulas (III-B), (III-C), (III-D) or (III-E) or a stereoisomer thereof, or a pharmaceutically acceptable salt thereof, or in combination with dexamethasone, or in combination with a proteasome inhibitor and dexamethasone, for use in the treatment of multiple myeloma.

In a fifth aspect, disclosed herein is a method of treating multiple myeloma in a subject, said method comprising administering to the subject a therapeutically effective amount of a Bcl-2 inhibitor of Formulas (III-B), (III-C), (III-D) or (III-E) or a stereoisomer thereof, or a pharmaceutically acceptable salt thereof, or in combination with dexamethasone, or in combination with a proteasome inhibitor and dexamethasone, for use in the treatment of multiple myeloma.

In a sixth aspect, disclosed herein is a use of a pharmaceutical composition in the manufacture of a medicament for use in the treatment of multiple myeloma, said pharmaceutical combination comprising a Bcl-2 inhibitor of Formulas (III-B), (III-C), (III-D) or (III-E) or a stereoisomer thereof, or a pharmaceutically acceptable salt thereof, and dexamethasone.

In a seventh aspect, disclosed herein is a use of a pharmaceutical composition in the manufacture of a medicament for use in the treatment of multiple myeloma, said pharmaceutical combination comprising a Bcl-2 inhibitor of Formulas (III-B), (III-C), (III-D) or (III-E) or a stereoisomer thereof, or a pharmaceutically acceptable salt thereof, a proteasome inhibitor, and dexamethasone.

In an embodiment of each of the above aspects, the Bcl-2 inhibitor is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl) pyrrolidin-1-yl)-7-azaspiro[3.5]nonan-7-yl)benzamide (Compound 1) or a pharmaceutically acceptable salt thereof.

In an embodiment of each of the above aspects, the proteasome inhibitor is carfilzomib or bortezomib.

In one embodiment of each of the above aspects, the multiple myeloma is relapsed/refractory multiple myeloma (R/R MM).

In one embodiment of each of the above aspects, the multiple myeloma demonstrates cytogenetic abnormalities and/or gene expression changes. In some embodiments, the multiple myeloma is t (11;14) positive multiple myeloma.

In one embodiment of each of the above aspects, the Bcl-2 inhibitor is orally administrated. In some embodiments, the Bcl-2 inhibitor is administrated in combination with dexamethasone daily on a 21-day cycle. In some embodiments, the Bcl-2 inhibitor is administrated with dexamethasone and carfilzomib on a 28-day cycle.

In one embodiment of each of the above aspects, the Bcl-2 inhibitor is orally administrated at a dose of 40 to 640 mg once daily (QD). In some embodiments, the Bcl-2 inhibitor is administrated at a dose of 40 mg QD, 80 mg QD, 160 mg QD, 320 mg QD, or 640 mg QD.

In some embodiments, dexamethasone is administrated at a dose of 40 mg/m2 once weekly. In some embodiments, dexamethasone is administrated simultaneously with the Bcl-2 inhibitor.

In some embodiments, dexamethasone is carfilzomib is administrated at a dose of 56 mg/m2 or 70 mg/m2 once weekly. In some embodiments, carfilzomib is administrated simultaneously with the Bcl-2 inhibitor.

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein). In some embodiments, the subject is a human or a patient.

The terms “cancer” or “tumor” herein has the broadest meaning as understood in the art and refers to the physiological condition in mammals that is typically characterized by unregulated cell growth. In the context of the present disclosure, the cancer is not limited to a certain type or location.

The term “therapeutically effective amount” as herein used, refers to the amount of a Bcl-2 inhibitor that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

### FIGURE DESCRIPTION

FIG. 1: FIG. 1A shows the effect of single and combo treatment of Compound 1 and dexamethasone at 0.1 mg/kg, and FIG. 1B shows the effect of single and combo treatment of Compound 1 and dexamethasone at 1 mg/kg.

FIG. 2: PK properties of Compound 1 in KMS-12-PE human MM subcutaneous xenograft model.

FIG. 3: Effect of Compound 1 in combination with dexamethasone on body weight in KMS-12-PE human MM subcutaneous xenograft model.

FIG. 4: Early results in Patients with R/R multiple myeloma harboring t (11, 14) mutation, ORR, overall response rate; sCR, stringent complete response; CR, complete response; VGPR, very good partial response; MR, minimal response; PR, partial response; DLT, dose-Limiting Toxicity; TEAE, treatment emergent adverse event.

### DETAILED DESCRIPTION OF THE DISCLOSURE

Disclosed herein are methods of treating multiple myeloma with a Bcl-2 inhibitor, in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof, or in combination with dexamethasone, or in combination with a proteasome inhibitor and dexamethasone.

### Bcl-2 Inhibitor

The Bcl-2 inhibitor in the present disclosure is a compound represented by the following Formulas (III-B), (III-C), (III-D) or (III-E),

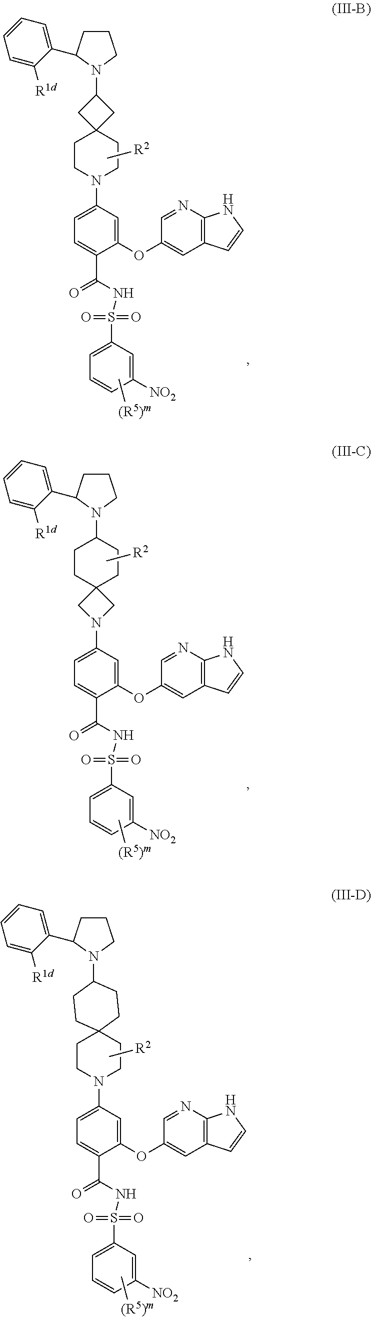

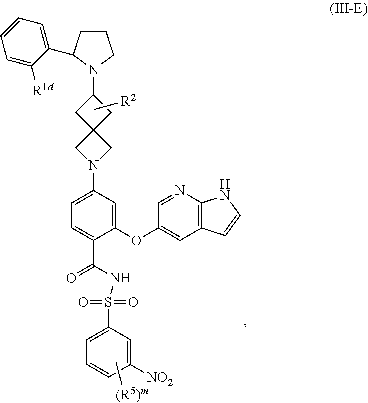

or a pharmaceutically acceptable salt thereof, or a stereoisomer thereof,
wherein,
R², at each occurrence, is independently selected from the group consisting of hydrogen, halogen, or -C₁₋₈alkyl optionally substituted with halogen;
R^{1d}, at each occurrence, is independently halogen, -C₁₋₈alkyl, -C₂₋₈alkenyl, -C₂₋₈alkynyl, cycloalkyl, heterocyclyl, aryl, heteroaryl, oxo, —CN, —NO₂, —OR^{Ba}, —SO₂R^{Ba}, —COR^{Ba}, —CO₂R^{Ba}, —CONR^{Ba}R^{Bb}, —C(═NR^{Ba})NR^{Bb}R^{Bc}, —NR^{Ba}R^{Bb}, —NR^{Ba}COR^{Bb}, —NR^{Ba}CONR^{Bb}R^{Bc}, —NR^{Ba}CO₂R^{Bb}, —NR^{Ba}SONR^{Bb}R^{Bc}, —NR^{Ba}SO₂NR^{Bb}R^{Bc}, or —NR^{Ba}SO₂R^{Bb}; wherein said -C₁₋₈alkyl, -C₂₋₈alkenyl, -C₂₋₈alkynyl, cycloalkyl, heterocyclyl, aryl or heteroaryl are each independently optionally substituted with 1 to 4 substituents R^{Bd},
R^{Ba}, R^{Bb}, and R^{Bc}, are each independently hydrogen, -C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, -C₂₋₈alkynyl, cycloalkyl, heterocyclyl, aryl, or heteroaryl is optionally substituted with halogen, hydroxy, —NH₂ or —N(C₁₋₆alkyl)₂, -C₁₋₈alkyoxy, cycloalkyl, heterocyclyl, aryl, or heteroaryl;
R^{Bd}, at each occurrence, is independently hydrogen, halogen, oxo, —CN, —NO₂, —C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, -C₂₋₈alkynyl, cycloalkyl, heterocyclyl, aryl, or heteroaryl is optionally substituted with halogen, hydroxy, -C₁₋₈alkyoxy, cycloalkyl, heterocyclyl, aryl, or heteroaryl;
m is an integer of 1-4;
R⁵ is -L⁵-CyC,
   Wherein L⁵is a direct bond, -(CR^{a}R^{b})ₜ-, —(CR^{a}R^{b})ₜ₋₁-(CR^{c}═CR^{d})-(CR^{a}R^{b})ᵥ₋₁-, -(CR^{a}R^{b})ₜ₋₁-(C≡C)-(CR^{a}R^{b})ᵥ₋₁-, —O—, —S—, —S(O)—, —SO₂-, —C(O)—, C(O)O—, —OC(O)—, —NR^{a}-, —C(O)NR^{a}-, —NR^{a}C(O)—, —NR^{a}C(O)O—, —NR^{a}C(O)NR^{b}-, —SO₂NR^{a}-, —NR^{a}SO₂-, —NR^{a}S(O)₂NR^{b}-, —NR^{a}S(O)NR^{b}-, —C(O)NR^{a}SO₂-, —C(O)NR^{a}SO—, or —C(═NR^{a})NR^{b}-, wherein t and v, at each occurrence, are independently a number of 1 to 7, and one or two CR^{a}R^{b} moieties in -(CR^{a}R^{b})ₜ-, -(CR^{a}R^{b})ₜ₋₁-(CR^{c}═CR^{d})-(CR^{a}R^{b})ᵥ₋₁-, -(CR^{a}R^{b})ₜ₋₁-(C≡C)-(CR^{a}R^{b})ᵥ₋₁- are un-replaced or replaced with one or more moieties selected from O, S, SO, SO₂, C(O) and NR^{a};
CyC is cycloalkyl, heterocyclyl, aryl, or heteroaryl, each of which is optionally substituted with one or two substituents R^{5a};
R^{5a}, at each occurrence, is independently selected from hydrogen, halogen, cyano, oxo, —NO₂, —OR^{5b}, —SR^{5b}, —NR^{5b}R^{5c}, —COR^{5b}, —SO₂R^{5b}, —C(═O)OR^{5b}, —C(═O)NR^{5b}R^{5c}, —C(═NR^{5b})NR^{5c}R^{5d}, —N(R^{5b})C(═O)R^{5c}, —N(R^{5b})C(═O)OR^{5c}, —N(R^{5b})C(O)NR^{5c}R^{5d}, —N(R^{5b})S(O)NR^{5c}R^{5d}, —N(R^{5b})S(O)₂NR^{5c}R^{5d}, —NR^{5b}SO₂R^{5c}, -C₁₋₈alkyl, -C₂₋₈alkenyl, -C₂₋₈alkynyl, -cycloalkyl, heterocyclyl, aryl, or heteroaryl, each of said -C₁₋₈alkyl, -C₂₋₈alkenyl, -C₂₋₈alkynyl, -cycloalkyl, heterocyclyl, aryl, or heteroaryl is optionally substituted with one or two substituents R^{5e};
   wherein R^{5b}, R^{5c}, and R^{5d} are each independently hydrogen, -C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, C₂₋₈alkynyl, -cycloalkyl, heterocyclyl, aryl, or heteroaryl is optionally substituted with one or two substituents R^{5e};
   R^{5e}, at each occurrence, is independently selected from hydrogen, halogen, cyano, oxo, —NO₂, —OR^{5f}, —SR^{5f}, —NR^{5f}R^{5g}, —COR^{5f}, —SO₂R^{5f}, —C(═O)OR^{5f}, —C(═O)NR^{5f}R^{5g}, —C(═NR^{5f})NR^{5g}R^{5h}, —N(R^{5f})C(═O)R^{5g}, —N(R^{5f})C(═O)OR^{5g}, —N(R^{5f})C(O)NR^{5g}R^{5h}, —N(R^{5f})S(O)NR^{5g}R^{5h}, —N(R^{5f})S(O)₂NR^{5g}R^{5h}, —NR^{5f}SO₂R^{5g}, -C₁₋₈alkyl, -C₂₋₈alkenyl, -C₂₋₈alkynyl, -cycloalkyl, heterocyclyl, aryl, or heteroaryl;
      R^{5f}, R^{5g}, and R^{5h} are each independently hydrogen, -C₁₋₈alkyl, -C₂₋₈alkenyl, -C₂₋₈alkynyl, cycloalkyl, heterocyclyl, aryl, or heteroaryl;
or, two adjacent R⁵ on the phenyl ring together with the phenyl ring form a benzo ring, said ring is optionally substituted with halogen, oxo, cyano, —NO₂, —OR⁵ⁱ, —SR⁵ⁱ, —NR⁵ⁱR^{5j}, —COR⁵ⁱ, —SO₂R⁵ⁱ, —C(═O)OR⁵ⁱ, —C(═O)NR⁵ⁱR^{5j}, —C(═NR⁵ⁱ)NR⁵ⁱR^{5k}, —N(R⁵ⁱ)C(═O)R^{5j}, —N(R⁵ⁱ)C(═O)OR^{5j}, —N(R⁵ⁱ)C(O)NR⁵ⁱR^{5k}, —N(R⁵¹)S(O)NR⁵ⁱR^{5k}, —N(R⁵ⁱ)S(O)₂NR⁵ⁱR^{5k}, —NR⁵ⁱSO₂R^{5k}, -C₁₋₈alkyl, -C₂₋₈alkenyl, -C₂₋₈alkynyl, -cycloalkyl, heterocyclyl, aryl, or heteroaryl;
   R⁵ⁱ, R^{5j}, and R^{5k} are independently hydrogen, -C₁₋₈alkyl, -C₂₋₈alkenyl, -C₂₋₈alkynyl, cycloalkyl, heterocyclyl, aryl, or heteroaryl, each of said -C₁₋₈alkyl, -C₂₋₈alkenyl, -C₂₋₈alkynyl, cycloalkyl, heterocyclyl, aryl, or heteroaryl is optionally substituted with halogen, hydroxy or -C₁₋₈alkyoxy;
R^{a}, R^{b}, R^{c}, and R^{d} at each occurrence, are independently hydrogen, -C₁₋₈alkyl, -C₂₋₈alkenyl, -C₂₋₈alkynyl, cycloalkyl, heterocyclyl, aryl, or heteroaryl, said -C₁₋₈alkyl, -C₂₋₈alkenyl, -C₂₋₈alkynyl, cycloalkyl, heterocyclyl, aryl, or heteroaryl are each independently substituted with —CN, halogen, —NO₂, —NR^{e}R^{f}, oxo, —OR^{e}, or —SR^{e}; and
wherein R^{e} and R^{f} are each independently hydrogen, C₁₋₈alkyl, C₁₋₈alkoxy-C₁₋₈alkyl-, C₂₋₈alkenyl, C₂₋₈alkynyl, cycloalkyl, aryl, heterocyclyl, or heteroaryl.

In some embodiments, R² is hydrogen.

In some embodiments, R^{1d}, when substituted on the phenyl group at position 2 of ring B (including the aziridin-1-yl, azetidin-1-yl, pyrrolidin-1-yl, pyrrolidin-2-yl, piperidin-1-yl, azepan-1-yl, or azocan-1-yl, preferably the pyrrolidin-1-yl group), is independently halogen, -C₁₋₈alkyl, -C₂₋₈alkenyl, -C₂₋₈alkynyl, cycloalkyl, heterocyclyl, aryl, heteroaryl, —CN, —OR^{Ba}, —SO₂R^{Ba}, —CONR^{Ba}R^{Bb}, —NO₂, —NR^{Ba}R^{Bb} —NR^{Ba}COR^{Bb}, or —NR^{Ba}SO₂R^{Bb}, wherein said -C₁₋₈alkyl, -C₂₋₈alkenyl, -C₂₋₈alkynyl, cycloalkyl, heterocyclyl, aryl or heteroaryl are each independently optionally substituted with 1 to 4 substituents R^{Bd} as defined as with Formulas (III-B), (III-C), (III-D) or (III-E)., preferably 1 or 2 substituents R^{Bd} as defined as with Formulas (III-B), (III-C), (III-D) or (III-E). In another aspect, one R^{1d} is at position 2 of the phenyl ring at position 2 of ring B.

In some embodiments, R^{1d} is methyl, ethyl, isopropyl, propyl or methoxymethyl, or two methyl at the position of the phenyl ring; or propenyl; or cyclopropyl, cyclobutyl, cyclopentyl, or cyclohexyl; or ethoxy or isopropoxy; or amino or dimethylamino.

In some embodiments, the 2-(2-substituted phenyl) pyrrolidin-1-yl moiety in Formulas (III-B), (III-C), (III-D) or (III-E), is selected from the group consisting of:

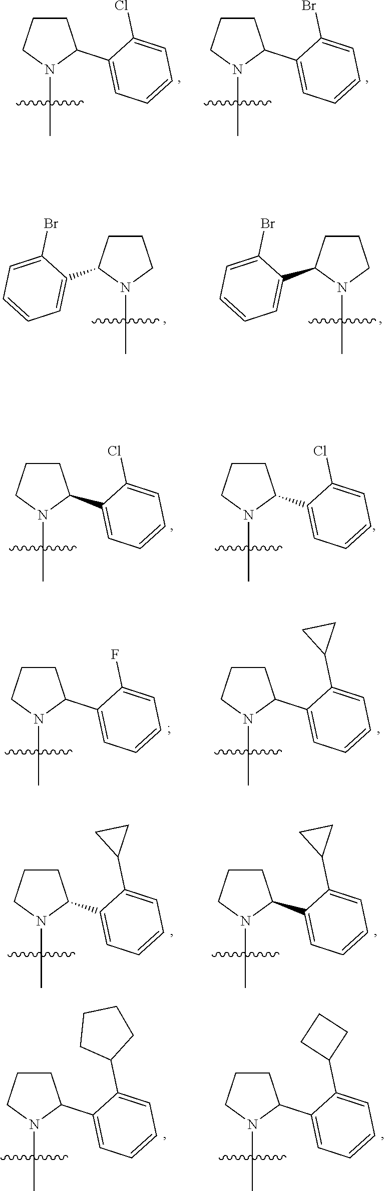

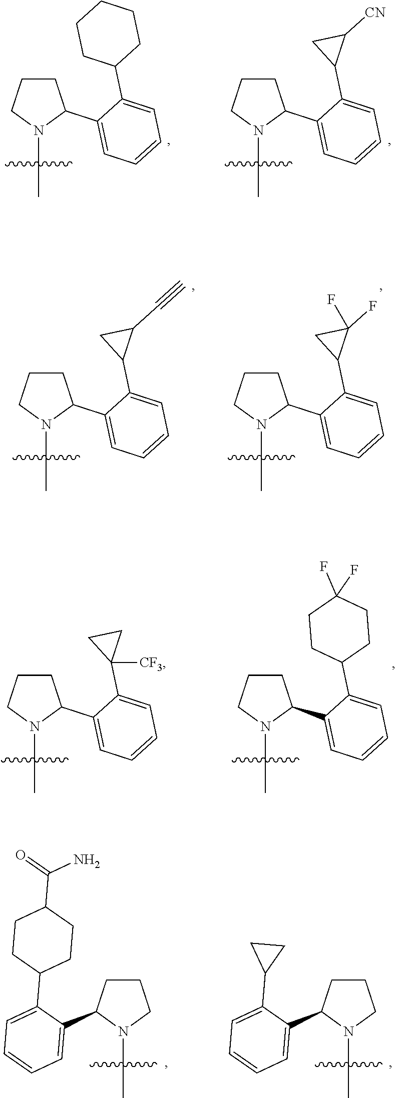

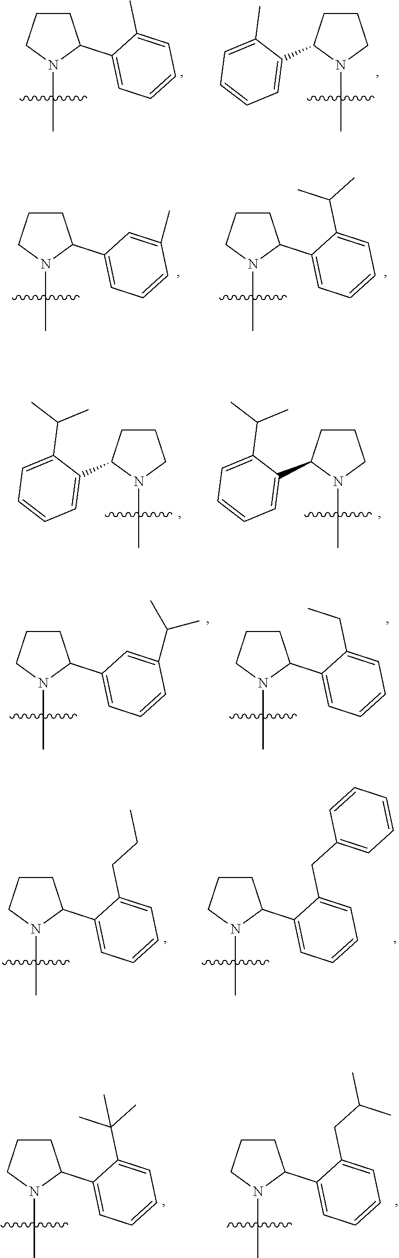

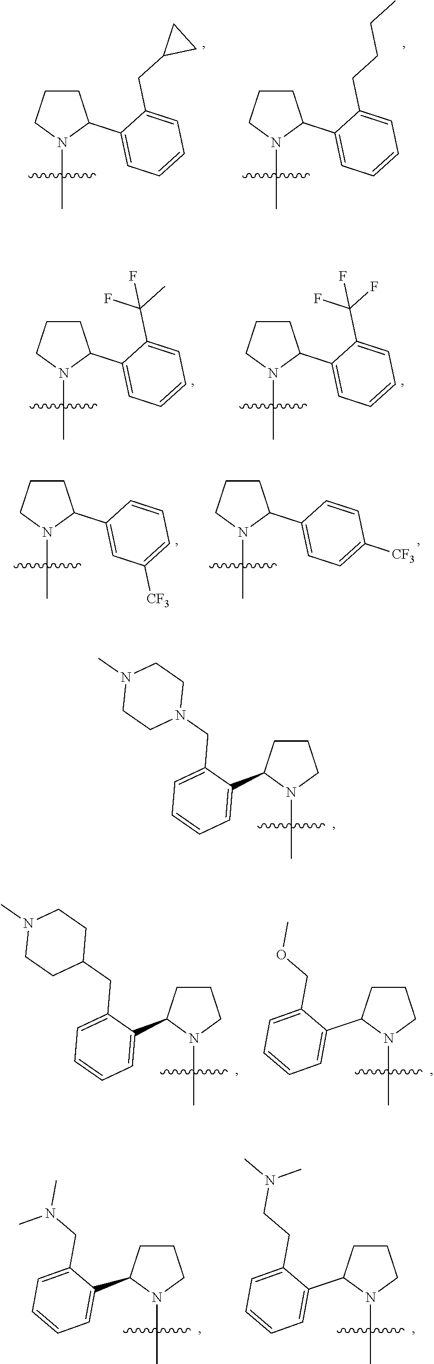

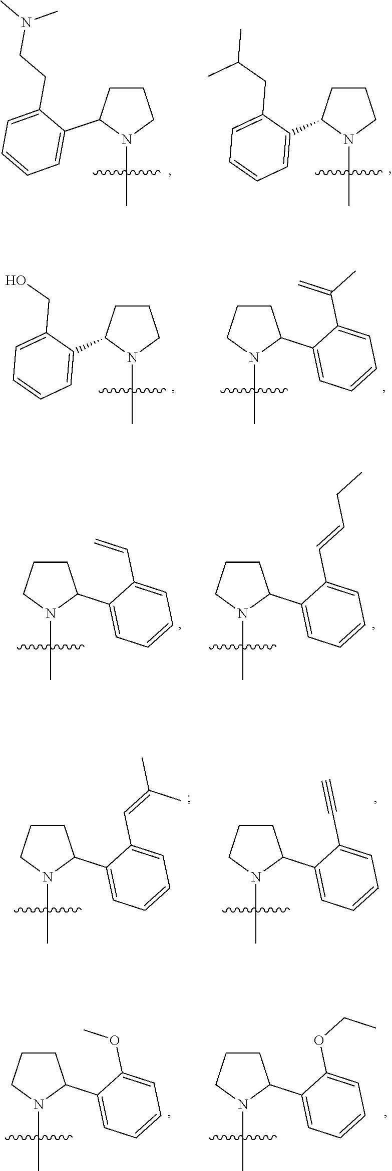

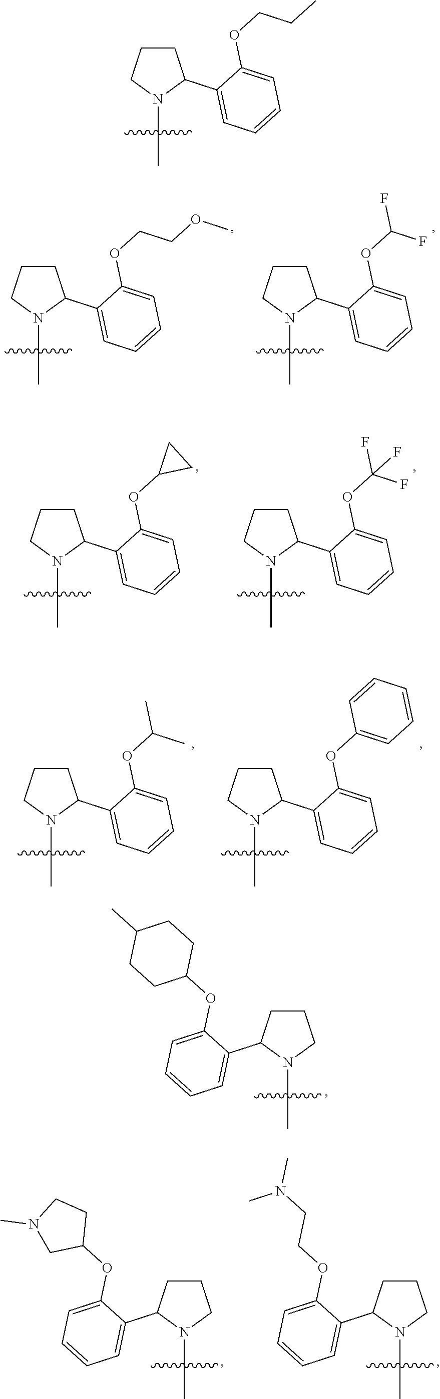

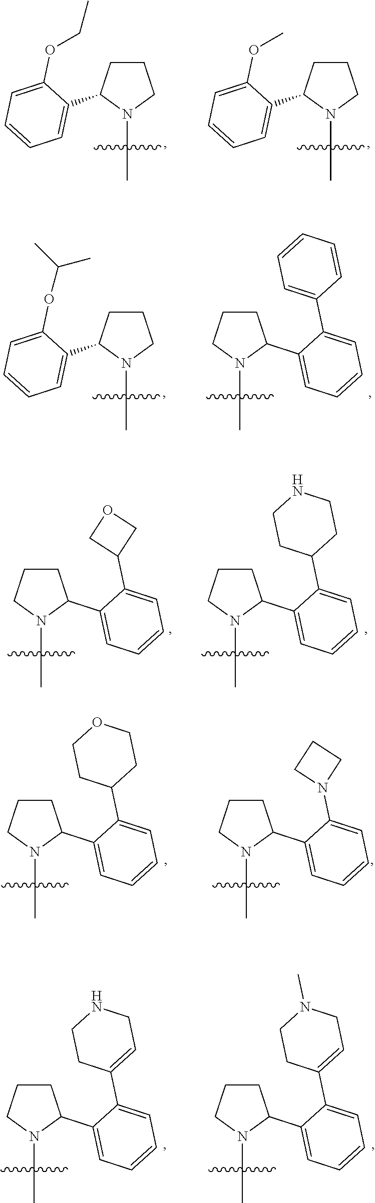

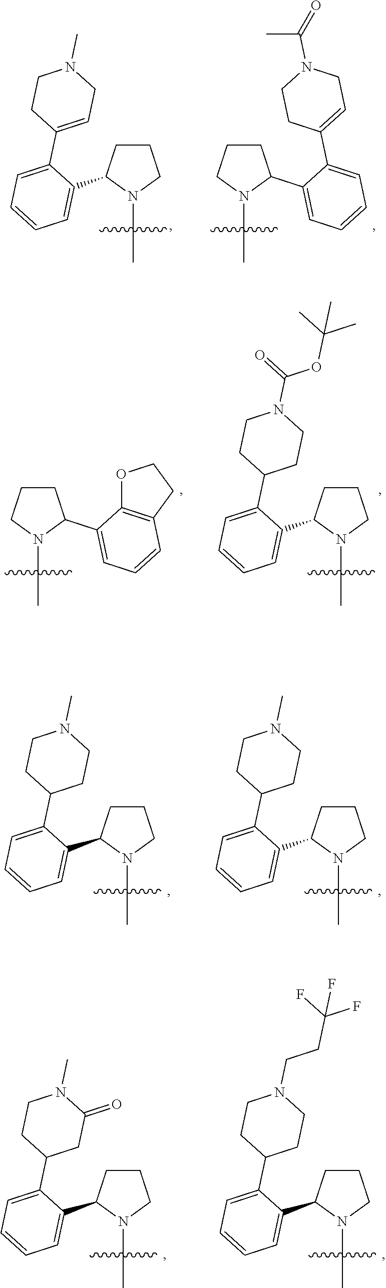

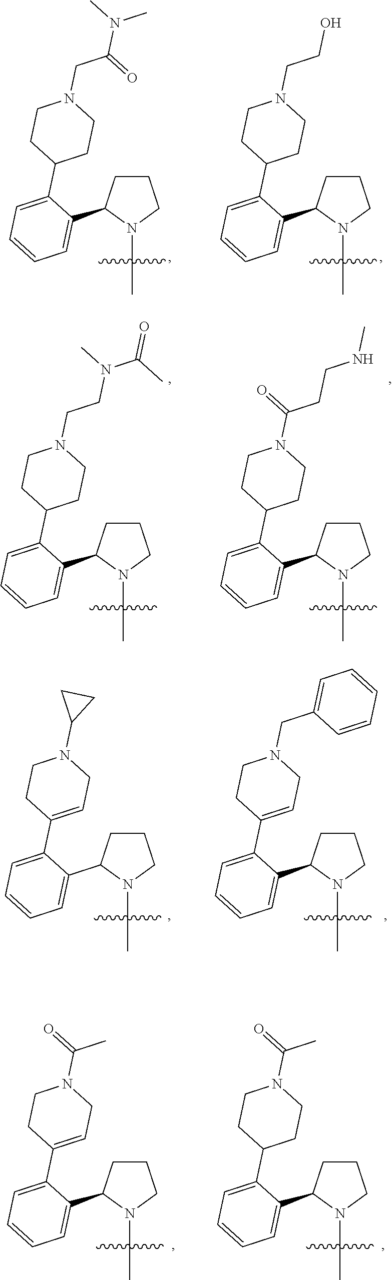

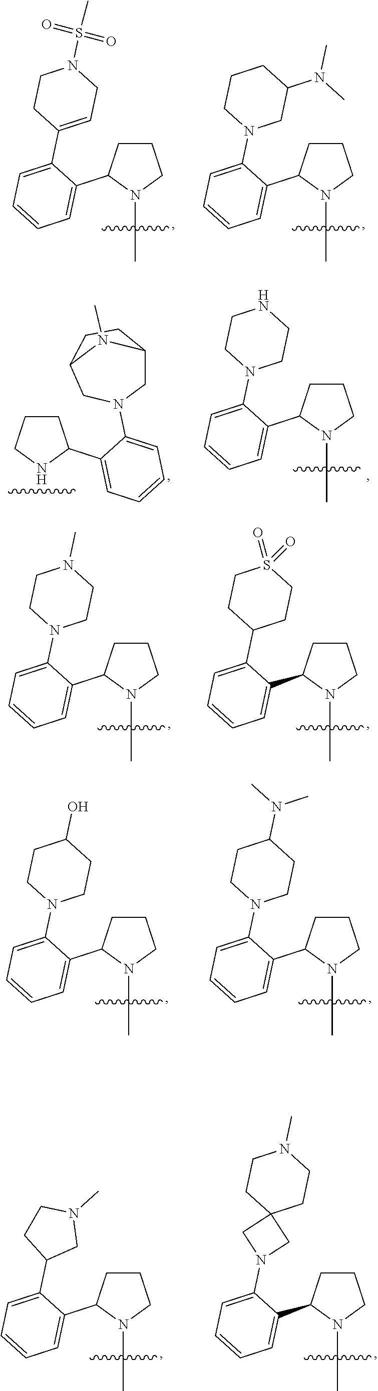

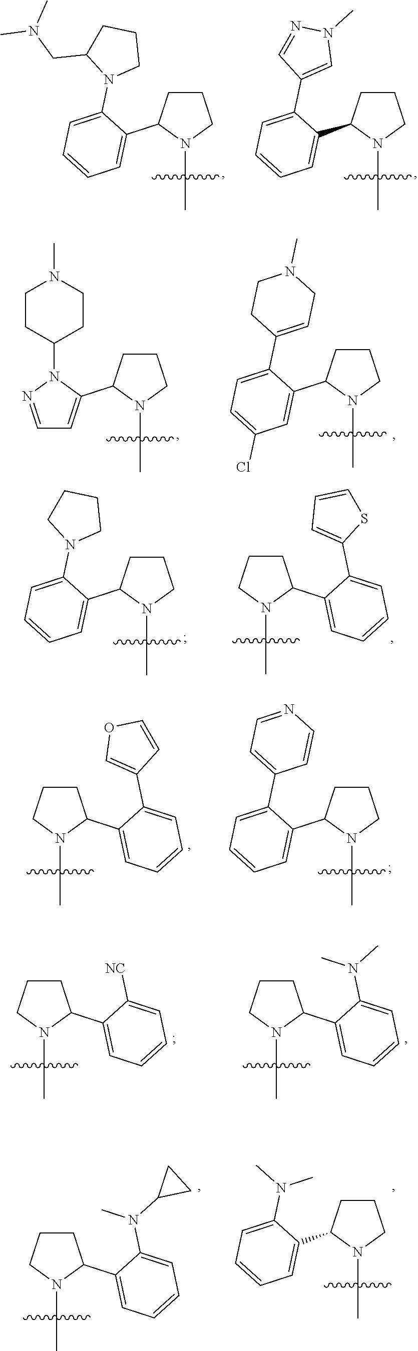

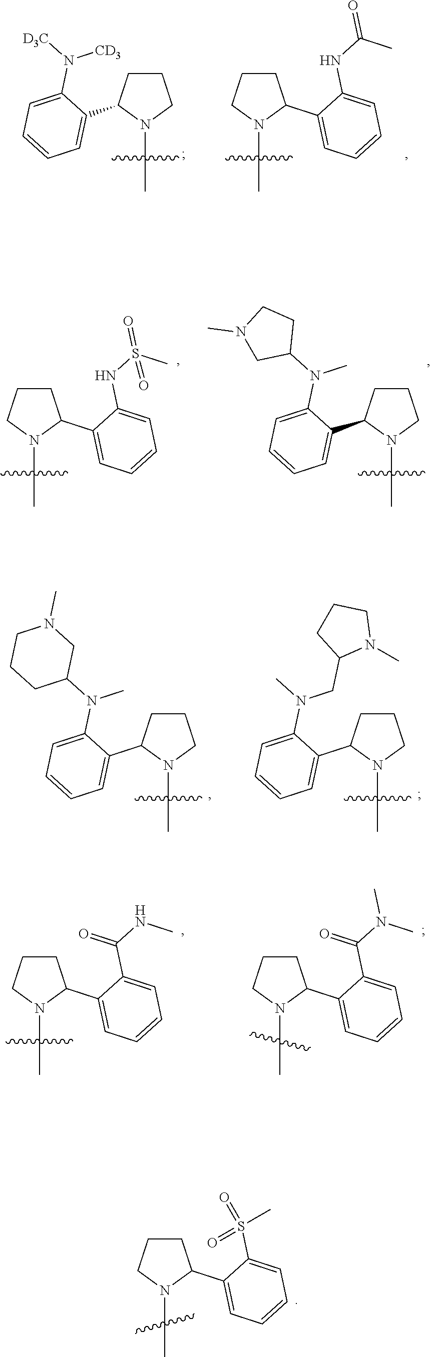

In some embodiments, m is 1; and L⁵ is a direct bond, -(CR^{a}R^{b})-or —NR^{a}-, wherein t is a number of 1 to 7, and one or two CR^{a}R^{b} moieties in -(CR^{a}R^{b})- are un-replaced or replaced with one or more moieties selected from O and NR^{a}, wherein R^{a} and R^{b} are defined with Formulas (III-B), (III-C), (III-D) or (III-E).

In some embodiments, L⁵ is a direct bond, -(CR^{a}R^{b})₁₋₄-, —O-(CR^{a}R^{b})₁₋₃-, —NH-(CR^{a}R^{b})₁₋₃, or —NH—, wherein R^{a} and R^{b} are defined as with Formulas (III-B), (III-C), (III-D) or (III-E), so that the -L⁵-CyC moiety is CyC, -(CR^{a}R^{b})₁₋₄-CyC, —O-(CR^{a}R^{b})₁₋₃-CyC, —NH-(CR^{a}R^{b})₁₋₃-CyC, or —NH—CyC, respectively. More preferably, L⁵ is a direct bond, —(CH₂)₁₋₄-, —O—(CH₂)₁₋₃-, —NH-(CR^{a}R^{b})-(CH₂)₂-, or —NH—, wherein R^{a} is hydrogen and R^{b} is C₁₋₈alkyl optionally substituted with phenyl-S- so that the -L⁵-CyC moiety is CyC, —(CH₂)₁₋₄-CyC, —O—(CH₂)₁₋₃-CyC, —NH-(CR^{a}R^{b})-(CH₂)₂-CyC, or —NH—CyC, respectively. More preferably, L⁵ is a direct bond, —CH₂-, —O—CH₂-, —NH—CH₂-, or —NH— so that the -L⁵-CyC moiety is CyC, —CH₂—CyC, —O—CH₂—CyC, —NH—CH₂—CyC, or —NH—CyC, respectively.

In some embodiments, CyC is cycloalkyl, or heterocyclyl, each of which is optionally substituted with one or two substituents R^{5a};
R^{5a} is independently selected from hydrogen, halogen, cyano, oxo, —OR^{5b}, —NR^{5b}R^{5c}, —COR^{5b}, —SO₂R^{5b}, -C₁₋₈alkyl, -C₂₋₈alkynyl, -cycloalkyl, or heterocyclyl, each of said -C₁₋₈alkyl, and heterocyclyl is optionally substituted with one or two substituents R^{5e} which is selected from hydrogen, halogen, cyano, —OR⁵, -C₁₋₈alkyl, -cycloalkyl, or heterocyclyl;
   wherein R^{5b}, and R^{5c} are each independently hydrogen, -C₁₋₈alkyl or heterocyclyl, said -C₁₋₈alkyl is optionally substituted with one or two substituents R^{5e} which is hydrogen, —NR^{5f}R^{5g}, or -cycloalkyl;
      R^{5f} and R^{5g} are each independently hydrogen or -C₁₋₈alkyl;
or, two adjacent R⁵ on the phenyl ring together with the phenyl ring form a benzo ring, said ring is optionally substituted with heteroaryl.

In some embodiments, CyC is cycloalkyl selected from monocyclic C₃₋₈cycloalkyl or bridged cycloalkyl

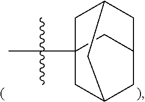 [SMILES: *C12CC3CC(CC(C3)C1)C2]

each of which is optionally substituted with one or two substituents R^{5a}. preferably, CyC is cyclopentyl or cyclohexyl, each of which is optionally substituted with one or two substituents R^{5a}.

In some embodiments, CyC is heterocyclyl selected from:
a) monocyclic 4 to 9-membered heterocyclyl groups containing one nitrogen or oxygen or sulfur heteroatom as a ring member;
b) monocyclic 4 to 9-membered heterocyclyl groups containing two heteroatoms selected from oxygen, sulfur and nitrogen as ring members; and
c)5 to 20-membered spiro heterocyclyl comprising one or two heteroatoms selected from nitrogen, sulfur and oxygen as ring members,
   each of which is optionally substituted with one or two R^{5a}.

In some embodiments, CyC is monocyclic 4 to 6-membered heterocyclyl groups containing one nitrogen or oxygen or sulfur heteroatom as the ring member. More preferably, Cyc is selected from oxetanyl, tetrahydrofuranyl, tetrahydropyranyl, azetidinyl, pyrrolidinyl, and piperdinyl. Even more preferably, CyC is selected from □oxetan-2-yl, Oxetan-3-yl, tetrahydrofuran-4-yl, tetrahydrofuran-2-yl, tetrahydrofuran-3-yl, tetrahydropyran-2-yl, tetrahydropyran-3-yl, tetrahydropyran-4-yl, azetidin-3-yl, azetidin-2-yl, pyrrolidin-2-yl, pyrrolidin-3-yl, piperdin-4-yl, piperdin-2-yl, and piperdin-3-yl.

In some embodiments, CyC is a monocyclic 6-membered heterocyclyl group containing two heteroatoms selected from oxygen and nitrogen as ring members. More preferably, CyC is dioxanyl, morpholino, morpholinyl, or piperzinyl. Even more preferably 1,3-dioxan-2-yl, 1,3-dioxan-4-yl, 1,4-dioxan-2-yl, morpholin-1-yl, morpholin-2-yl, or morpholin-3-yl.

In some embodiments, R^{5a} is independently selected from hydrogen, halogen, cyano, oxo, —OR^{5b}, —NR^{5b}R^{5c}, —COR^{5b}, —SO₂R^{5b}, -C₁₋₈alkyl, -C₂₋₈alkynyl, monocyclic C₃₋₈cycloalkyl, or monocyclic 4 to 9-membered heterocyclyl group containing one or two heteroatoms selected from nitrogen or oxygen or sulfur heteroatom as ring members, each of said -C₁₋₈alkyl and monocyclic 4 to 9-membered heterocyclyl group is optionally substituted with one or two substituents R^{5e}; preferably, cycloalkyl as R^{5a} is C₃₋₆cycloalkyl; more preferably cyclopropyl; preferably, heterocyclyl as R^{5a} is 4 to 6-membered heterocyclyl groups containing one or two heteroatoms selected from nitrogen or oxygen or sulfur heteroatom as ring members; more preferably, heterocyclyl as R^{5a} is oxetanyl, tetrahydrofuranyl, tetrahydropyranyl, piperzinyl, or morpholinyl; even more preferably, heterocyclyl as R^{5a} is oxetan-3-yl, tetrahydrofuran-3-yl, tetrahydro-2H-pyran-4-yl, or morphin-4-yl.

In some embodiments, heterocyclyl as R^{5e} is a monocyclic 4 to 9-membered heterocyclyl group containing one or two heteroatoms selected from nitrogen or oxygen or sulfur heteroatom as ring members.

In some embodiments, heterocyclyl as R^{5e} is tetrahydro-pyran-4-yl.

In some embodiments, R^{5a} is—NR^{5b}R^{5c}, wherein R^{5b} is hydrogen, and R^{5c} is heterocyclyl.

In some embodiments, R^{5a} is—NR^{5b}R^{5c}, wherein R^{5b} is hydrogen, and R^{5c} is tetrahydro-pyran-4-yl.

In some embodiments, R^{5a} is—NR^{5b}R^{5c}, wherein R^{5b} and R^{5c} are each independently hydrogen or -C₁₋₆alkyl substituted with cycloalkyl, preferably-C₁₋₆alkyl substituted with monocyclic C₃₋₈cycloalkyl.

In some embodiments, R^{5a} is —OR^{5b} or —SO₂R^{5b}, wherein R^{5b} is hydrogen or C₁₋₈alkyl, preferably methyl.

In some embodiments, R^{5a} is —COR^{5b}, wherein R^{5b} is hydrogen or C₁₋₈alkyl optionally substituted with —NR^{5f}R^{5g}, wherein R^{5f} and R^{5g} are each independently hydrogen or C₁₋₈alkyl, preferably methyl.

In some embodiments, two adjacent R⁵ on the phenyl ring together with the phenyl ring form indazolyl which is substituted with tetrahydropyranyl.

In some embodiments, m is 1, and R5 is-L5-CyC selected from the group consisting of:

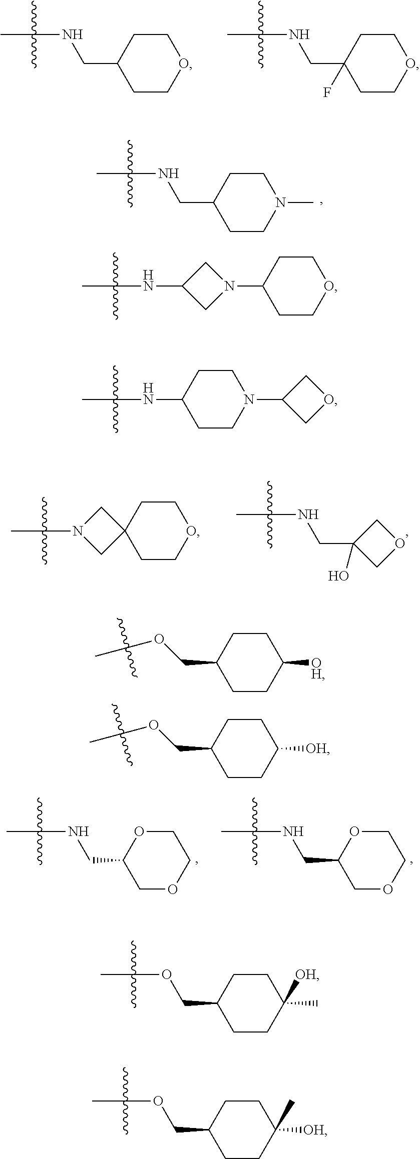

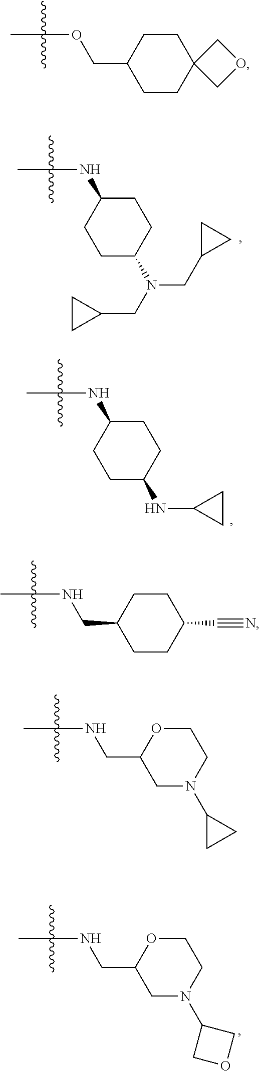 [SMILES: *OCC1CCC2(CC1)COC2 ; *N[C@H]1CC[C@H](N(CC2CC2)CC2CC2)CC1 ; *N[C@H]1CC[C@@H](NC2CC2)CC1 ; *NC[C@H]1CC[C@H](C#N)CC1 ; *NCC1CN(C2CC2)CCO1 ; *NCC1CN(C2COC2)CCO1]

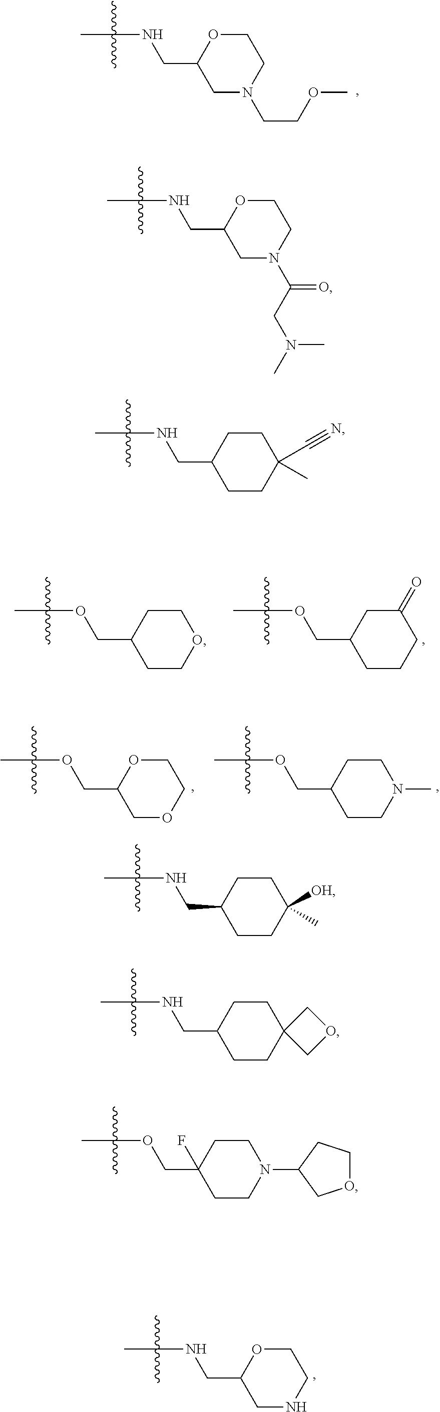

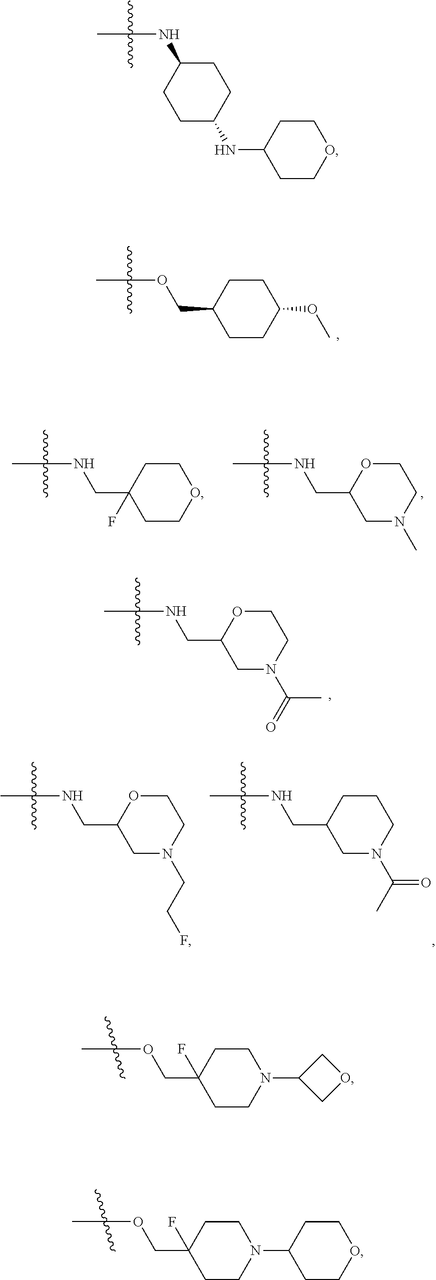

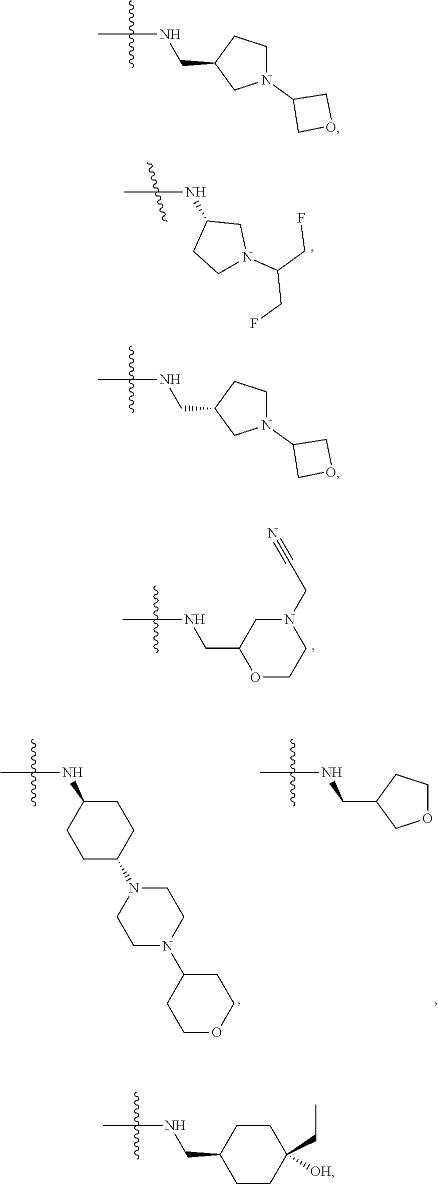

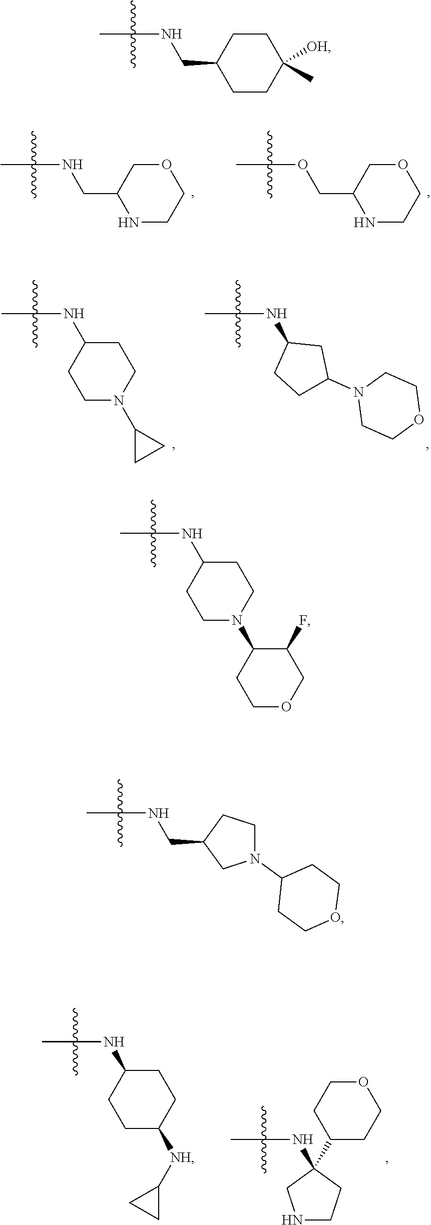

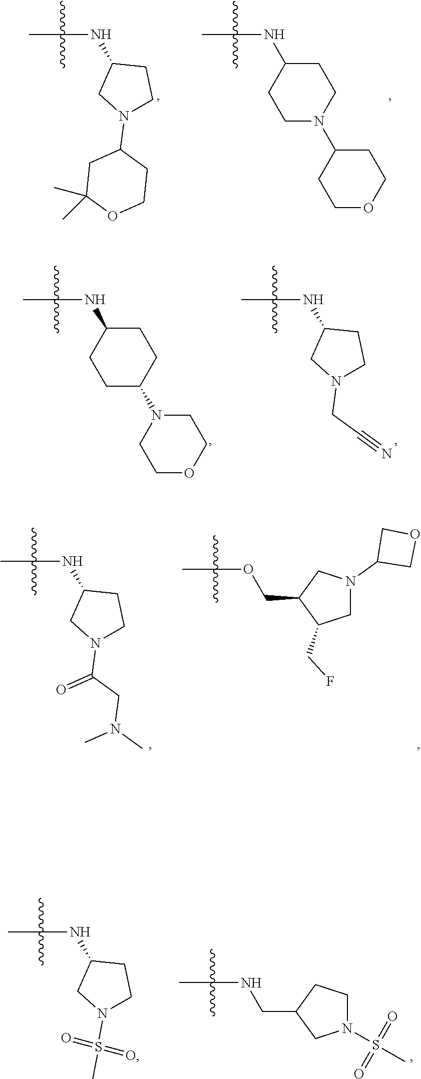

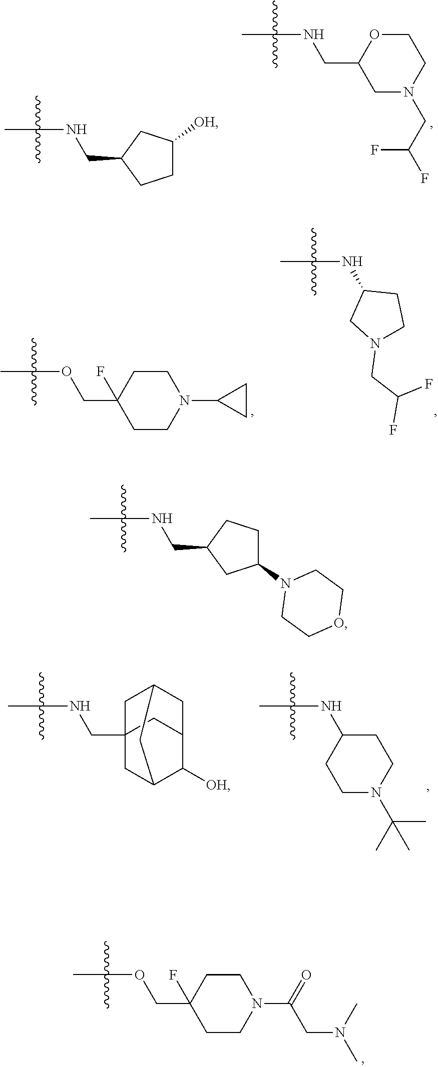

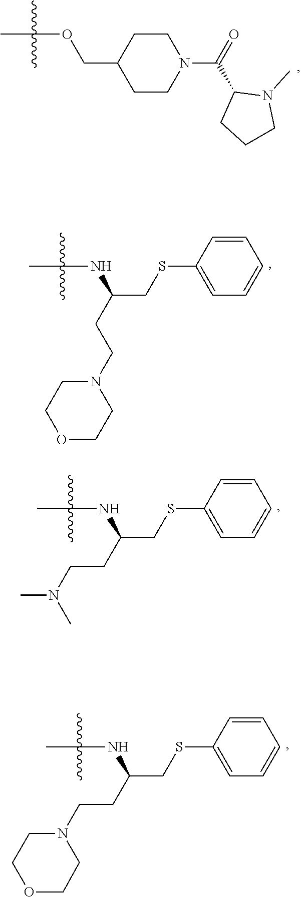

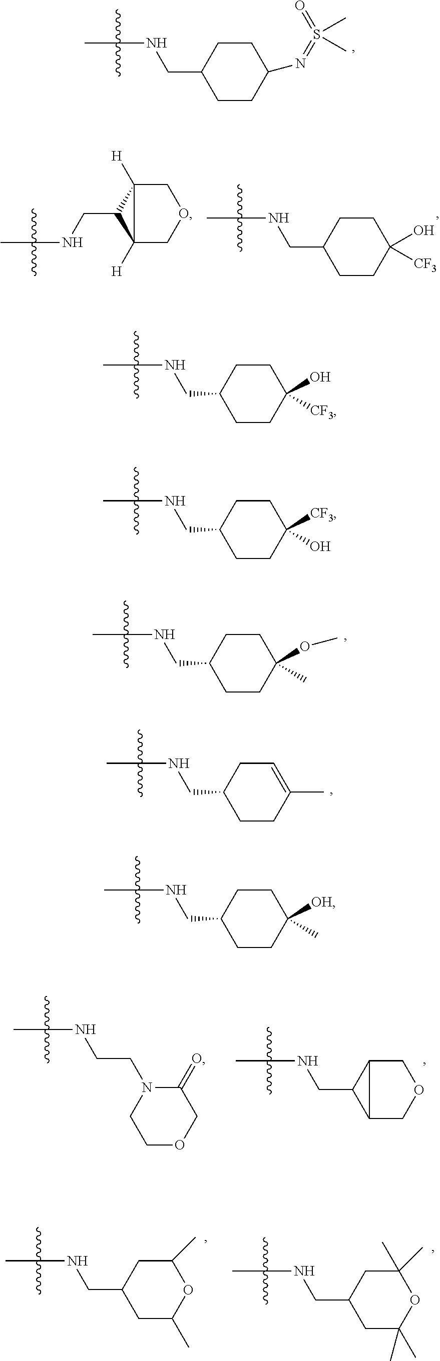

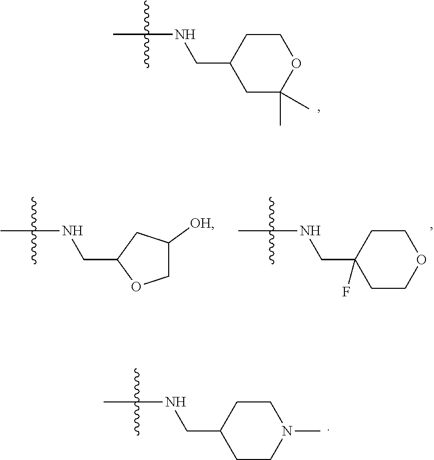

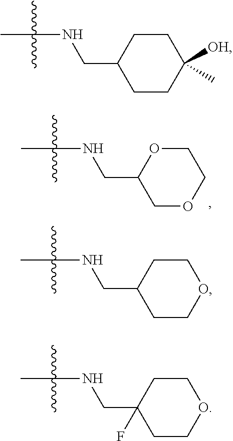

In some embodiments, m is 1and R⁵ is

In some embodiments, the Bcl-2 inhibitor in present disclosure is selected form the group consist of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2-isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis(methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1-yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;

2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl (oxo)-16-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl (3-nitro-4-(((tetrahydro-2H-pyran-4-yl)methyl)amino)phenyl)(oxo)-16-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide; 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide; (S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2-cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;; 2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
   or a pharmaceutically acceptable salt thereof, or a stereoisomer thereof.

In some embodiments, the Bcl-2 inhibitor in present disclosure is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound A) or a pharmaceutically acceptable salt thereof.

### Preparation of Bcl-2 Inhibitors

All the Bcl-2 inhibitors having Formulas (III-B), (III-C), (III-D) or (III-E), including 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1), can be prepared by the method disclosed in international publication WO2019/210828A1.

### Preparation of Compound 1

Step 1:2,2-dimethoxy-7-azaspiro[3.5]nonane Hydrochloride

To the solution of tert-butyl 2-oxo-7-azaspiro[3.5]nonane-7-carboxylate (500 g, 2.09 mol) in MeOH (750 mL) and EA (750 mL)was added conc. HCl acid (350 mL, 4.18 mol)at room temperature and stirred for 4 hours. After concentrated in vacuum, MeOH (750 mL)was added into the residue and then the resulting mixture was concentrated in vacuum (repeated this work-up twice). The brown residue was suspended in EA (1250 mL) and stirred for 1 hour. The solid precipitation was filtered and dried in vacuum to afford the title product as an off-white powder (350 g, yield: 76.0%). 1H NMR (400 MHZ, DMSO-d₆) δ ppm: 3.03 (s, 6 H), 2.96-2.89 (m, 4 H), 1.93 (s, 4 H), 1.74-1.67 (m, 4 H). MS (ESI, m/e) [M+1]⁺ 186.0.

Step 2: methyl 2-((1H-pyrrolo[2.3-b]pyridin-5-yl)oxy)-4-(2,2-dimethoxy-7-azaspiro[3.5]nonan-7-yl)benzoate

The mixture of methyl 2-((1H-pyrrolo[2,3-b]pyridin-5-yl)oxy)-4-fluorobenzoate (100 g), 2,2-dimethoxy-7-azaspiro[3.5]nonane hydrochloride (116 g, 1.5 eq.) and DBU (160 g, 3.0 eq.)in NMP (500 mL)was stirred for 16 hours at 85° C. After the reaction was completed, the mixture was cooled to 50±5° C. and citric acid in water (2%, 5 L)was added drop-wise into the system under stirring. After filtered, the cake was collected and dissolved with DCM (1.5 L). The solution of crude product was washed with citric acid in water (2%, 1.5 L), saturated aq. NaHCO₃ (1.5 L) and 15% aq. NaCl (1.5 L), and then dried over anhydrous Na₂SO₄. Silica gel (100 g)was added into the solution of the crude product under stirring and then filtered. The filtrate was concentrated to 300 mL. MTBE (500 mL)was poured into the system. After stirred for 2 hours, the cake was collected after filtration and was dried in vacuum to give an off-white solid (192 g, yield: 72.1%). ¹H NMR (400 MHZ, DMSO-d₆) δ ppm: 11.63 (s, 1H), 8.00 (d, J=2.4 Hz, 1H), 7.76 (d, J=9.2 Hz, 1H), 7.47 (t, J=3.2 Hz, 1H), 7.42 (d, J=2.4 Hz, 1H), 6.79 (dd, J=2.4 Hz, J=9.2 Hz, 1H), 6.39-6.36 (m, 2H), 3.64 (s, 3H), 3.17-3.12 (m, 4H), 3.01 (s, 6H), 1.86 (s, 4H), 1.54-1.50 (m, 4H). MS (ESI, m/e)[M+1]⁺ 451.9.

Step 3: methyl 2-((1H-pyrrolo[2,3-b]pyridin-5-yl)oxy)-4-(2-oxo-7-azaspiro[3.5]nonan-7-yl)benzoate

To the solution of methyl 2-((1H-pyrrolo[2,3-b]pyridin-5-yl)oxy)-4-(2,2-dimethoxy-7-azaspiro[3.5]nonan-7-yl)benzoate (176 g, 0.39 mol)in DCM (2 L) was added diluted HCl acid (1M, 1.5 L) and stirred for overnight. After the reaction was completed, the mixture was cooled to 10° C. and was adjusted to pH =8-9 with aqueous NaOH solution (4 M) under stirring. The organic phase was separated and washed with 15% aq. NaCl (1 L), then washed with H₂O (1 L). After the organic phase was concentrated to 500 mL, MTBE (1 L) was poured into the solution and then the system was concentrated to 500 mL (repeated this work-up 3 times). The resulting system was stirred for 0.5 hour. After filtration, the cake was collected and then dried in vacuum to obtain the tittle product as a white solid (152 g, yield: 96.23%). ¹H NMR (400 MHZ, DMSO-d₆) δ ppm: 11.64 (s, 1H), 8.02 (d, J=2.4 Hz, 1H), 7.78 (d, J=9.2 Hz, 1H), 7.47 (t, J=3.2 Hz, 1H), 7.44 (d, J=2.4 Hz, 1H), 6.83 (dd, J=2.4 Hz, J=9.2 Hz, 1H), 6.43 (d, J=2.4 Hz, 1H), 6.38-6.36 (m, 1H), 3.65 (s, 3H), 3.24-3.21 (m, 4H), 2.80 (s, 4H), 1.70-1.67 (m, 4H). MS (ESI, m/e) [M+1]⁺ 405.9.

Step 4: (S)-tert-butyl 2-(2-(prop-1-en-2-yl)phenyl)pyrrolidine-1-carboxylate

To a mixture of(S)-tert-butyl 2-(2-bromophenyl)pyrrolidine-1-carboxylate (50 g, 153.3 mmol) and 4,4,5,5-tetramethyl-2-(prop-1-en-2-yl)-1,3,2-dioxaborolane (38.6 g, 229.9 mmol)in dioxane (500 mL) and H₂O (50 mL) was added Cs₂CO₃ (100 g, 305 mmol) and Pd (dppf)Cl₂ (6.6 g, 7.5 mmol). The mixture was stirred at 100° C. for 8 hours. TLC showed the reaction was completed. The mixture was concentrated in vacuum. The residue was purified by column chromatography on silica gel (eluent: PE/EA (v/v)=100/1 to 10/1)to obtain (S)-tert-butyl 2-(2-(prop-1-en-2-yl)phenyl)pyrrolidine-1-carboxylate (65 g, crude). The crude product was used directly in next step.

Step 5: (S)-tert-butyl 2-(2-isopropylphenyl)pyrrolidine-1-carboxylate

To a solution of (S)-tert-butyl 2-(2-(prop-1-en-2-yl)phenyl)pyrrolidine-1-carboxylate (30 g, 104.39 mmol)in MeOH (500 mL) was added Pd/C (10 g, 10%) and the mixture was stirred at 20° C. under H₂ (15 Psi) for 12 hours. TLC showed the reaction was completed. The mixture was filtered and the filtrate was concentrated in vacuum to give(S)-tert-butyl 2-(2-isopropylphenyl)pyrrolidine-1-carboxylate (60 g, crude), which was used in next step without further purification. ¹H NMR (400 MHZ, CDCl₃) δ ppm: 7.39-6.90 (m, 4H), 5.36-5.04 (m, 1H), 3.77-3.52 (m, 2H), 3.20-3.17 (m, 1H), 2.47-2.24 (m, 1H), 1.96-1.65 (m, 3H), 1.54-1.38 (m, 2H), 1.31-1.22 (m, 8H), 1.17 (s, 7H).

Step 6: (S)-2-(2-isopropylphenyl)pyrrolidine Hydrochloride

To a solution of tert-butyl 2-(2-isopropylphenyl)pyrrolidine-1-carboxylate (55 g, 190 mmol) in DCM (50 mL) was added HCl in 1,4-dioxane (4 M, 142 mL, 570 mmol) dropwise at room temperature. The mixture was stirred at room temperature for overnight. The mixture was concentrated in vacuum. The resulting residue was slurried with EA (100 mL) and then filtered, dried in vacuum to give(S)-2-(2-isopropylphenyl)pyrrolidine hydrochloride 26 g (yield: 60.4%). ¹H NMR (400 MHZ, DMSO-d₆) δ ppm: 9.93 (s, 1H), 8.81 (s, 1H), 7.63-7.57 (m, 1H), 7.41-7.34 (m, 2H), 7.32-7.24 (m, 1H), 4.91-4.75 (m, 1H), 3.47-3.35 (m, 1H), 3.31-3.25 (m, 1H), 2.40-2.21 (m, 1H), 2.19-1.86 (m, 3H), 1.25 (d, J=6.7 Hz, 3H), 1.17 (d, J=6.7 Hz, 3H). MS (ESI, m/e)[M+1]⁺ 190.0.

Step 7: methyl(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate

A mixture of (S)-2-(2-isopropylphenyl)pyrrolidine hydrochloride (120 g, 0.535 mole) and methyl 2-((1H-pyrrolo[2,3-b]pyridin-5-yl)oxy)-4-(2-oxo-7-azaspiro[3.5]nonan-7-yl)benzoate (218 g, 0.509 mole) in DCM (2.2L) was charged into a reactor. The temperature was controlled blow 30° C. and NaBH(OAc)₃ (216 g, 1.018 mole) was added into the reactor in 5-6 portions. Then the reaction mixture was stirred at room temperature and monitored by TLC. After the starting material ketone was consumed completely, the mixture was adjusted to pH=4˜5 with diluted HCl acid (0.5 M). The separated organic phase was washed with H₂O (600 mL×2) and then washed with aq. NaHCO₃ (600 mL×2), saturated aq. NaCl (600 mL). The organic phase was collected, then dried over anhydrous Na₂SO₄ and concentrated. 256 g off-white solid was obtained as the crude product, which was used in the next step directly. MS (ESI, m/e)[M+1]⁺ 579.0.

Step 8: (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic Acid

To a solution of methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate (105 g, 181.7 mmol)in THF (525 mL) and MeOH (525 mL) was added aq. NaOH (3.5 M). It was stirred at room temperature overnight. After THF and MeOH were removed in vacuum, 3.5 L of water was added into the residue. The resulting mixture was adjusted to pH=5˜6 with 3 N HCl acid at room temperature with stirring. The precipitate was filtered and dried in vacuum to give the product as a white solid (102.4 g, yield: 99%). ¹H NMR (400 MHz, DMSO-d₆) δ ppm: 12.13 (s, 1H), 11.58 (s, 1H), 7.95 (s, 1H), 7.67 (d, J=8.0 Hz, 1H), 7.56-7.40 (m, 2H), 7.35 (s, 1H), 7.27-7.04 (m, 3H), 6.68 (d, J=8.0 Hz, 1H), 6.32 (s, 2H), 3.62 (s, 1H), 3.32-3.26 (m, 1H), 3.10-3.04 (m, 4H), 2.35-2.30 (m, 1 H), 2.9-2.15 (m, 1 H), 1.74-1.64 (m, 4H), 1.52-1.37 (m, 6H), 1.28-1.06 (m, 6H). MS (ESI, m/e)[M+1]⁺ 564.9.

Step 9: 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide

A mixture of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid (44 g, 78 mmol), 4-((((1r,4r)-4-hydroxy-4-methylcyclohexyl)methyl)amino)-3-nitrobenzenesulfonamide (26.8 g, 78 mmol), TEA (15.7 g, 156 mmol), EDCI (19.4 g, 101 mmol) and DMAP (19 g, 156 mmol)in anhydrous DCM (880 mL) was stirred overnight at room temperature. The reaction was monitored by HPLC. After starting material of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid was consumed completely, the reaction mixture was heated to ˜35° C. and N¹,N¹-dimethylethane-1,2-diamine (17.2 g, 195 mmol) was added in one portion. The reaction was stirred for another 12 hours. The mixture was washed twice with 10 wt % aq. AcOH solution (300 mL×2) and then washed with saturated aq. NaHCO₃ (300 mL×2). The organic layer was collected and concentrated to about 90 mL. 22 g of silica gel was added and stirred for 2 hours. After filtration, 180 mL EA was added into the filtrate at reflux and further stirred for 5 hours. After the mixture was cooled to room temperature, the precipitate was filtered and then the wet cake was washed twice with EA (180 mL). After drying in vacuum at 80-90° C., the desired compound was obtained (48 g, yield: 69.5%). ¹H NMR (DMSO-d₆) δ ppm: 11.65 (s, 1H), 11.11 (br, 1H), 8.58-8.39 (m, 2H), 8.00 (d, J=2.8 Hz, 1H), 7.74 (d, J=8.8 Hz, 1H), 7.57-7.37 (m, 4H), 7.30-7.10 (m, 3H), 7.00 (d, J=9.2 Hz, 1H), 6.65 (d, J=1.2 Hz, 1H), 6.35 (s, 1H), 6.17 (s, 1H), 4.24 (s, 1H), 3.39-3.20 (m, 5H), 3.04-2.88 (m, 4H), 2.23 (s, 1H), 1.94-1.47 (m, 11H), 1.44-1.26 (m, 7H), 1.19 (d, J=8.0 Hz, 3H), 1.14 (d, J=8.0 Hz, 3H), 1.10 (s, 4H). MS (ESI, m/e) [M+1]⁺ 889.9.

### Methods of Treatment

In one aspect, the present disclosure provides a method of treating multiple myeloma. In certain aspects, the method comprises administering to a patient a therapeutically effective amount of Compound 1, or in combination with dexamethasone, or in combination with a proteasome inhibitor and dexamethasone.

Compound 1 can be administered by any suitable means, including oral, parenteral,

intrapulmonary, and intranasal, and, if desired for local treatment, intralesional administration. Dosing can be by any suitable route. Various dosing schedules including but not limited to single or multiple administrations over various time-points, bolus administration, and pulse infusion are contemplated herein.

Compound 1 would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Compound 1 is optionally formulated with one or more agents currently used to prevent or treat the disorder in question. The effective amount of such other agents depends on the amount of Compound 1 in the formulation, the type of disorder or treatment, and other factors discussed above.

For the prevention or treatment of disease, the appropriate dosage of Compound 1 will depend on the type of disease to be treated, the severity and course of the disease, whether Compound 1 is administered for preventive or therapeutic purposes, previous therapy, the patient's clinical history and response to Compound 1, and the discretion of the attending physician. Compound 1 is suitably administered to the patient at one time or over a series of treatments.

### EXAMPLES

The present invention is further exemplified, but not limited to, by the following examples that illustrate the invention.

### Example 1

### KMS-12-PE Xenograft Model Study

### 1. Study Materials

### 1.1 Test Article(s)

Dexamethasone (HPLC purity: ≥98%, MW=392.46).

### 1.1.1 Preparation of Vehicle

Compound 1 was formulated for oral dosing in 60% (v/v) phosal 50 PG, 30% (v/v) PEG 400, and 10% (v/v) ethyl alcohol). The vehicle was prepared according to the following procedures. To make 10 mL vehicle, 3 mL PEG 400, 6 mL phosal 50 PG, and 1 mL ethyl alcohol were added into a clean tube, vortexed for 1 min and then sonicated for 3 min to make a clear solution. The solution was stored at 2-8° C. until use.

### 1.1.2 Preparation of Test Article

Compound 1 was formulated for oral dosing at 1.5 mg/mL with vehicle according to the following procedures: 1) Weigh an appropriate amount of test article into a clean tube. 2) Add the vehicle (approximately 60% to 80% of total volume) into the tube using syringe. 3) Vortex the tube for 3 min and sonicate it for 10 min. 4) Repeat the above procedures until Compound 1 was completely dissolved. 5) Add the vehicle to reach the final volume.

Dexamethasone was firstly formulated for oral dosing at 1 mg/mL, and diluted to 0.1 mg/mL with saline.

### 1.2 Experimental System

65 female NCG mice were purchased from GemParmatech Co., Ltd. On the day of inoculation, mice were, 6-7 weeks of age in a weight range of 17.9-22.9 g. All animals were maintained under specific pathogen free (SPF) “full barrier” condition with free access to food and water. Mice were group-housed under a 12-h light: dark cycle (lights on at 08:00 am), temperature range at 22.3-24.8° C., humidity range at 45.2-65.8% in IVC cages. Animals were fed with completely granulated feed with Co₆₀ radio sterilization.

### 2. Experimental Procedures

### 2.1 Cell Culture

KMS-12-PE, a human MM cell line, was obtained from Deutsche Sammlung von Mikroorganismen und Zellkulturen (DSMZ, Catalog: ACC 606). KMS-12-PE cells were cultured in RPMI 1640 complete medium, supplemented with 20% (v/v) fetal bovine serum, and 100 μg/mL of penicillin and streptomycin. In this study, cells were used within 10 passages of recovery from liquid nitrogen. KMS-12-PE cells were maintained as suspension cultures at 37° C. in a 5% CO₂ atmosphere, being passaged twice weekly.

### 2.2 Cell Inoculation

On the day of inoculation, cell suspension was centrifuged for 5 min at 1,000 rpm and cell pellet was re-suspended in an appropriate volume of pre-cold PBS. Cells were counted in a hemocytometer and their viability was assessed using 0.4% trypan blue. Cells were re-suspended in pre-cold PBS at 3×10⁷ cells/mL and same volume of matrigel was added to give a final concentration of 1.5×10⁷ cells/mL. Resuspended cells were placed on ice prior to inoculation. The right front flank of each mouse was cleaned with 75% ethanol prior to cell inoculation. Each animal was injected subcutaneously with 3×10⁷ cells in 200 μL of cells suspension in the right front flank via a 1 mL LS 25GA syringe.

### 2.3 Animal Grouping and Administration

On day 4 after cell inoculation, animals were randomly assigned into 6 groups with 8 mice per group according to body weight and tumor volume (185.0-223.1 mm³). Mice were treated once daily (QD) with vehicle, dexamethasone at 0.1 or 1 mg/kg, or Compound 1 at 1.5 mg/kg for 17 days. Treatments were administered by oral gavage (p.o.) in a volume of 10 mL/kg body weight. Body weight was assessed immediately before dosing and volume dosed was adjusted accordingly.

### 2.4 Clinical Observation, Body Weight, and Tumor Volume

Tumor volume was measured twice weekly in two dimensions using a caliper (measurable since day 4 post treatment in this study). Body weights were recorded twice weekly. One individual was responsible for tumor measurement for the duration of the study. Mice were also monitored daily for clinical signs of toxicity. Mice were euthanized using carbon dioxide at the end of the study.

### 2.5 PK Analysis

On day 14 post treatment, blood samples (about 100 μL per mouse) were collected from the retro-orbital sinus under isoflurane/oxygen anesthesia at predose 0, 0.5, 2, 8, and 24 hours after dosing of Compound 1 (3 mice per time point, every mouse was used for 2 or 3 time points). The blood was transferred to the EDTA-K2 anticoagulation tube, and the plasma was collected by centrifugation at 5,600 rpm for 7 min and was kept frozen in −80° C. until analysis.

### 3. Data Analysis

The two-dimension diameters of tumor were determined using a caliper. Tumor volume was calculated using the formula: V=0.5×(a×b²) where a and b were the long and short diameters of the tumor, respectively.

Tumor growth inhibition (TGI) was calculated using the following formula:

$$\% TGI = 100 \times ( 1 - ( \frac{( treated t ) - ( treated {t}_{0} )}{( vehicle t ) - ( vehicle {t}_{0} )} ) )$$

Where, treated t=treated tumor volume at time t, treated t₀=treated tumor volume at time 0, vehicle t=vehicle tumor volume at time t, vehicle t₀=vehicle tumor volume at time 0.

For PK analysis, maximum plasma concentration (Cₘₐₓ) and time to peak (Tₘₐₓ) were got directly from the plasma concentration versus time graphs. The area under the plasma concentration-time curve (AUC) from time 0 to 24 hours post-dose (AUC₀₋₂₄ₕ) was calculated.

The tumor volume on day 17 post treatment was analyzed using ANOVA method on logarithmic scale in SAS Enterprise Guide version: 7.15 HF3. The following comparisons were made among treatment groups,

The combinations of Compound 1 at 1.5 mg/kg with dexamethasone at 0.1 mg/kg or 1 mg/kg were compared to the vehicle group, with Dunnett's multiple comparison test.

The combination therapy was compared to corresponding single agents. If the combination therapy shows improved anti-tumor activity versus both single agents, a significant combination effect is claimed (i.e., the EOHSA criterion). The EOHSA tests were adjusted by Holm's multiple comparison test. #p<0.05, ####p<0.0001.

Adjusted-p<0.05 was considered statistically significant.

### 4. Results

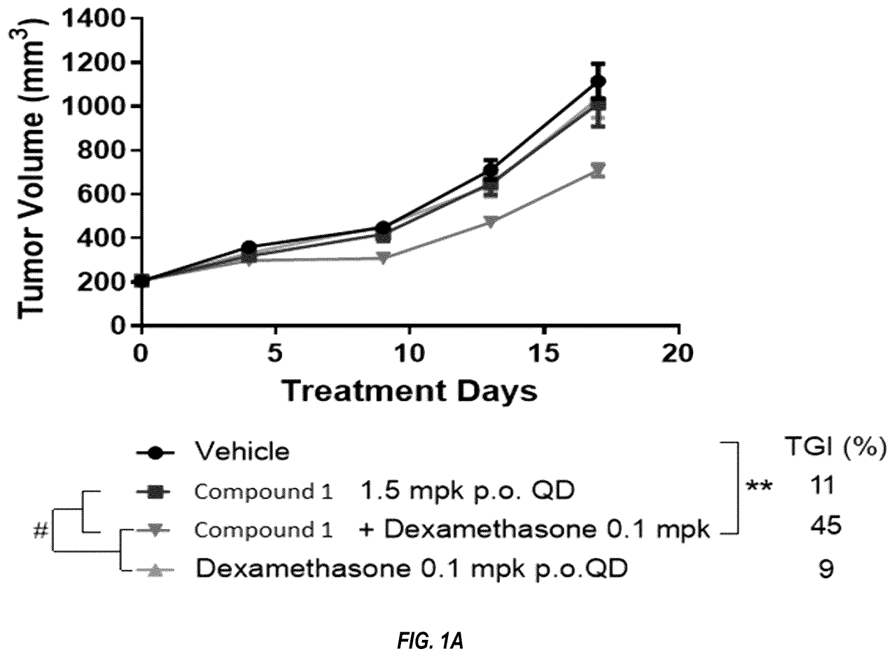

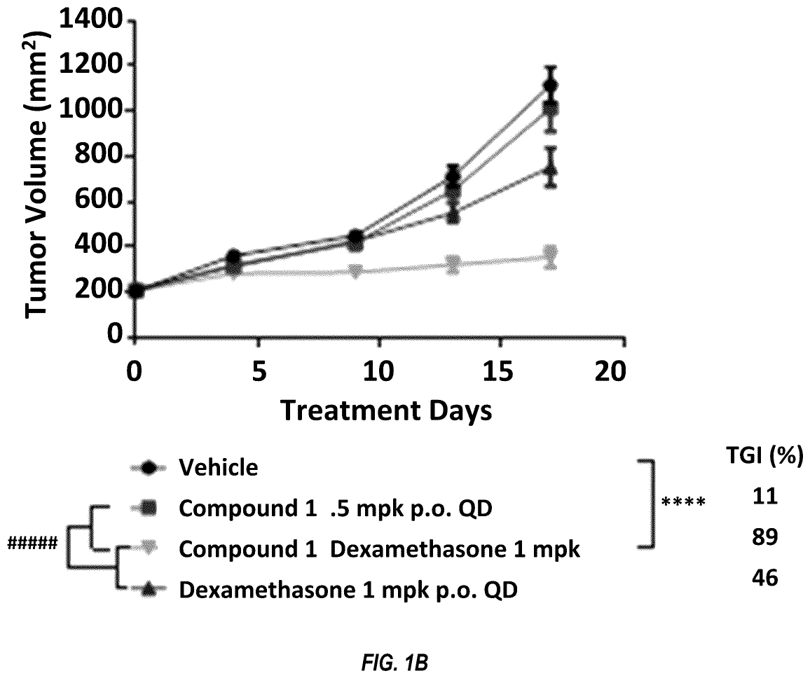

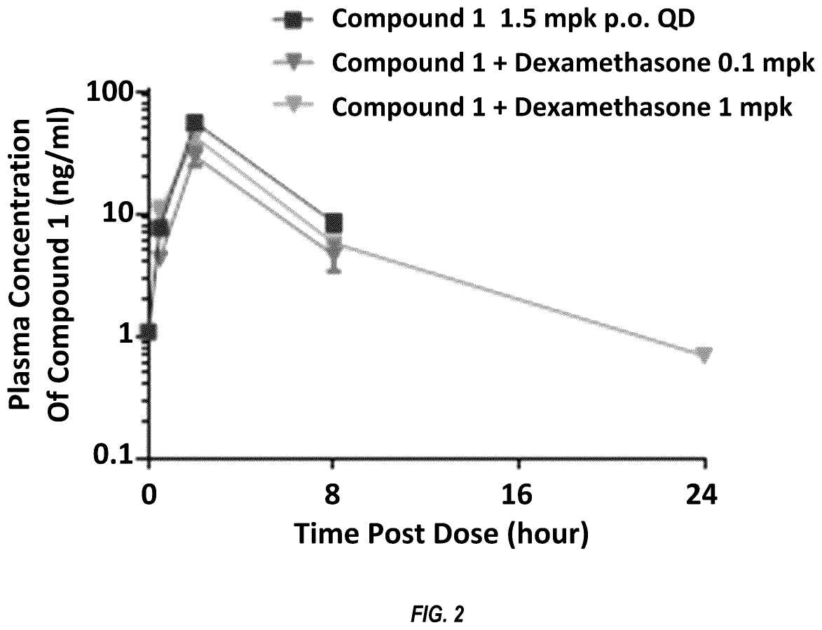

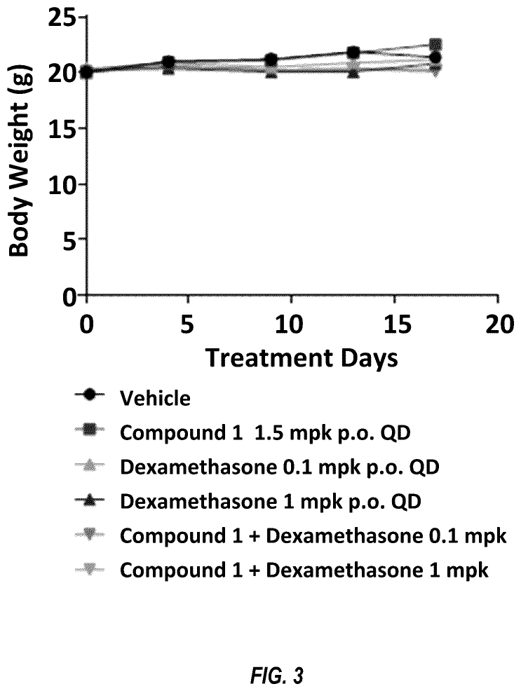

This study has evaluated the combinational effect of Compound 1 plus dexamethasone in the KMS-12-PE human MM subcutaneous xenograft model in NCG mice. The results were shown in FIG. 1A, 1B, FIG. 2, FIG. 3 and Table 1-Table 2.

FIG. 1 showed the effect of Compound 1 in combination with dexamethasone on tumor growth in KMS-12-PE human MM subcutaneous xenograft model. KMS-12-PE tumor cells (3×10⁷) were implanted subcutaneously in the female NCG mice. On day 4 post inoculation, mice were divided into 6 groups with 8 mice per group according to body weight and tumor volume and treated for 17 days as indicated. Tumor volume was measured twice a week. FIG. 1A and FIG. 1B showed the effect of single and combo treatment of Compound 1 and dexamethasone at 0.1 mg/kg (A) or at 1 mg/kg (B) on tumor growth. Data was presented as mean tumor volume+standard error of the mean (SEM) of 8animals in each group. The tumor volume on day 17 was analyzed using ANOVA method on logarithmic scale. Combination therapies were compared to the vehicle group with Dunnett's multiple comparison test (**p<0.01, ****p<0.0001). Combination effects were evaluated with EOHSA criterion, with Holm's multiple comparison test (#p <0.05, ####p<0.0001). Abbreviations: p.o., oral gavage; QD, once daily; TGI, tumor growth inhibition; mpk, mg/kg.

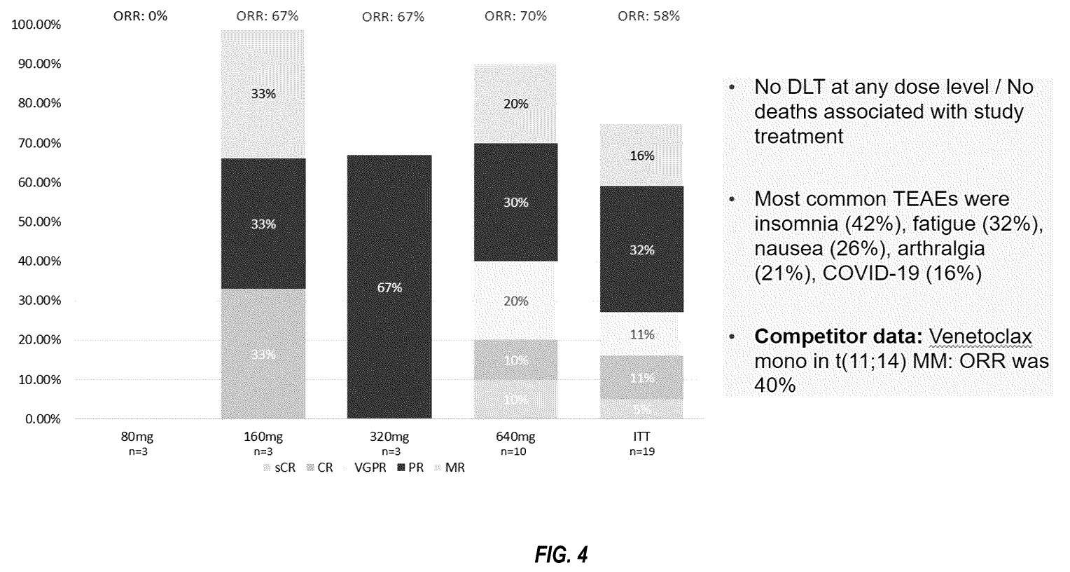

FIG. 4 showed the PK properties of Compound 1 in KMS-12-PE human MM subcutaneous xenograft model. KMS-12-PE tumor cells (3×10⁷) were implanted subcutaneously in the female NCG mice. On day 4 post inoculation, mice were divided into 6 groups with 8 mice per group according to body weight and tumor volume and treated for 17 days as indicated. On day 14 post treatment, blood samples were collected from the retro-orbital sinus under carbon dioxide/oxygen anesthesia at 0, 0.5, 2, 8, and 24 hours after dosing of Compound 1. Data was presented as mean plasma concentration±SEM of 3 animals in each time point. Abbreviations: p.o., oral gavage; QD, once daily; mpk, mg/kg.

FIG. 5 showed the effect of Compound 1 in combination with dexamethasone on body weight in KMS-12-PE human MM subcutaneous xenograft model. KMS-12-PE tumor cells (3×10⁷) were implanted subcutaneously in the female NCG mice. On day 4 post inoculation, mice were divided into 6 groups with 8 mice per group according to body weight and tumor volume and treated for 17 days as indicated. Body weight was measured twice a week since treatment. Data was presented as mean body weight±SEM of 8 animals in each group. Abbreviations: p.o., oral gavage; QD, once daily; mpk, mg/kg.

As a single agent, oral administration of Compound 1 at 1.5 mg/kg QD or dexamethasone at 0.1 mg/kg QD showed little inhibitory effects on tumor growth, with the TGI of 11% and 9% on day 17 post treatment, respectively (FIG. 1A and Table 1). Significantly improved anti-tumor activity was found with the combined treatment of Compound 1 and dexamethasone at 0.1 mg/kg, with the TGI of 45% (adjusted p<0.01). The combination therapy showed significantly improved anti-tumor activity versus both single agents, i.e., the combination effect was significant per EOHSA criterion (adjusted p<0.05) (FIG. 1A and Table 1).

Oral administration of dexamethasone at 1 mg/kg QD showed certain anti-tumor activity, with the TGI of 46% on day 17 (FIG. 1B and Table 1). A superior efficacy with a TGI of 89% was shown in mice orally administered with the combination of Compound 1 and dexamethasone at 1 mg/kg (adjusted p<0.0001). The combination effect was significant per EOHSA criterion (adjusted p<0.0001) (FIG. 1B and Table 1).

Besides, the exposure (AUC^{0-24h} and Cₘₐₓ) of Compound 1 at steady state in single treatment group was consistent with that in combo treatment (FIG. 2, Table 1 and Table). No obvious impact on body weight was observed in all treatment groups (FIG. 3).

To conclude, all the results indicate that the combination of Compound 1 and dexamethasone showed better anti-tumor activity than either single agent in the KMS-12-PE xenograft model.

TABLE 1
Efficacy and PK parameters of Compound 1 in combination with
dexamethasone in human KMS-12-PE xenograft model

| | Dose of | | | | PK Parameters of | |
|---|---|---|---|---|---|---|
| | Com- | Dose of | Tumor | | Compound 1 | |
| | pound | dexameth- | Volume (mm³) | TGI | Cₘₐₓ | AUC_{0-24 h} |
| | 1 | asone | (Mean ± SEM) | (%) | (ng/mL) | (ng*h/mL) |
| Treatment | (mg/kg) | (mg/kg) | Day 17 | Day 17 | Day 14 | |
| Vehicle p.o. QD | NA | NA | 1115.2 ± 80.0 | NA | NA | NA |
| Compound 1 1.5 | 1.5 | NA | 1011.7 ± 103.8 | 11 | 55.8 | 200 |
| mg/kg p.o. QD | | | | | | |
| Dexamethasone | NA | 0.1 | 1036.5 ± 88.8 | 9 | NA | NA |
| 0.1 mg/kg p.o. QD | | | | | | |
| Dexamethasone | NA | 1 | 696.0 ± 69.2 | 46 | NA | NA |
| 1 mg/kg p.o. QD | | | | | | |
| Compound 1 | 1.5 | 0.1 | 709.0 ± 28.6 | 45 | 29.5 | 107 |
| 1.5 mg/kg + | | | | | | |
| Dexamethasone | | | | | | |
| 0.1 mg/kg | | | | | | |
| Compound 1 | 1.5 | 1 | 304.4 ± 20.2 | 89 | 42.4 | 179 |
| 1.5 mg/kg + | | | | | | |
| Dexamethasone | | | | | | |
| 1 mg/kg | | | | | | |

Abbreviations: p.o., oral gavage; QD, once daily; TGI, tumor growth inhibition; NA, not applicable; SEM, standard error of the mean; Cₘₐₓ, maximum plasma concentration; AUC_{0-24 h}, AUC from time zero to 24 hours post-dose.

TABLE 2
Individual animal's plasma concentration (ng/mL) of Compound 1 at
indicated time points at the steady state (on day 14 post treatment)

| | Animal | PK Time | Sample | Time Post Treatment (h) | | | | |
|---|---|---|---|---|---|---|---|---|
| Treatment | ID | Point | ID | 0 | 0.5 | 2 | 8 | 24 |
| Compound 1 | 14 | 0, 0.5, and | 2-1 | 1.09 | NA | 62.8 | NA | BLOQ |
| 1.5 mg/kg | 31 | 24 h | 2-2 | BLOQ | NA | 40.7 | NA | BLOQ |
| p.o. QD | 34 | | 2-3 | BLOQ | NA | 63.9 | NA | BLOQ |
| | 40 | 2 and 8 h | 2-4 | NA | 8 | NA | 9.97 | NA |
| | 43 | | 2-5 | NA | 5.72 | NA | 9.73 | NA |
| | 52 | | 2-6 | NA | 9.29 | NA | 5.81 | NA |
| Compound 1 | 8 | 0, 0.5, and | 5-1 | BLOQ | NA | 36.7 | NA | BLOQ |
| 1.5 mg/kg + | 10 | 24 h | 5-2 | BLOQ | NA | 30.9 | NA | BLOQ |
| Dexameth- | 11 | | 5-3 | BLOQ | NA | 20.8 | NA | BLOQ |
| asone | 48 | 2 and 8 h | 5-4 | NA | 4.02 | NA | 3.18 | NA |
| 0.1 mg/kg | 57 | | 5-5 | NA | 5.2 | NA | 6.95 | NA |
| | 58 | | 5-6 | NA | 3.67 | NA | 3.59 | NA |
| Compound 1 | 4 | 0, 0.5, and | 6-1 | BLOQ | NA | 54.9 | NA | BLOQ |
| 1.5 mg/kg + | 18 | 24 h | 6-2 | BLOQ | NA | 35.8 | NA | 0.692 |
| Dexameth- | 19 | | 6-3 | BLOQ | NA | 36.4 | NA | BLOQ |
| asone | 23 | 2 and 8 h | 6-4 | NA | 14.4 | NA | 5.15 | NA |
| 1 mg/kg | 28 | | 6-5 | NA | 6.94 | NA | 7.02 | NA |
| | 32 | | 6-6 | NA | 9.04 | NA | 5.05 | NA |

Abbreviations: p.o., oral gavage; QD, once daily; BLOQ, below limit of quantitation; NA, not applicable.

### Example 2

### Clinical Study

### 1. Method

### Study Design/Objectives

### Part 1 (Dose Escalation)

A dose escalation study is conducted to evaluate the safety and tolerability of Compound 1 in combination with dexamethasone and dexamethasone plus carfilzomib in patients with relapsed/refractory (R/R) multiple myeloma (MM) and t (11;14). Also, the corresponding maximum tolerated dose (MTD)/maximum assessed dose (MAD), recommended Phase 2 dose (RP2D) and pharmacokinetics for Compound 1 in the combination therapies are to be observed.

Compound 1 plus dexamethasone: Patients with R/R MM and t (11;14) receive increasing doses of Compound 1 once daily with starting dose at 80 mg once daily, plus 40 mg of dexamethasone once weekly to determine the safety, tolerability, efficacy, and pharmacokinetics (PK). Upon completion of the dose-limiting toxicity (DLT) window (21 days) and depending on the number of evaluable patients and observed DLTs, the dose of Compound 1 may be de-escalated to 40 mg once daily, stay at 80 mg once daily, or be escalated to 160 mg once daily. Upon dose escalation to 160 mg once daily and provided there are no DLTs observed during the DLT window, the study may be conducted to escalate to a few higher dose levels (up to 640 mg once daily), or to insert some certain intermediate doses between 80 mg and 160 mg once daily.

One analysis will be conducted for the Compound 1 plus dexamethasone cohort when all patients from the corresponding dose escalation have completed the DLT observation window. The analysis for Compound 1 plus dexamethasone cohort will be to confirm the RP2D as well as to summarize the safety profile of Compound 1 in combination with dexamethasone based on all available data.

Compound 1 plus dexamethasone plus carfilzomib: The combination of Compound 1 plus dexamethasone plus carfilzomib is to be evaluated, according to RP2D of Compound 1 in combination with dexamethasone. The initial dose of Compound 1 in combination with dexamethasone and carfilzomib will be one dose level below the previously determined RP2D defined for the Compound 1 plus dexamethasone cohort. The initial starting dose of carfilzomib will be 56 mg/m2 per week. The dose of dexamethasone will remain at 40 mg once weekly. There are initially 3 dose combinations planned for Compound 1 plus carfilzomib plus dexamethasone, including,
1) Compound 1 at RP2D-1 dose level+carfilzomib at 56 mg/m2 per week+dexamethasone,
2) Compound 1 at RP2D-1 dose level+carfilzomib at 70 mg/m2 per week+dexamethasone, and,
3) Compound 1 at RP2D+carfilzomib at 70 mg/m2 per week+dexamethasone.

RP2D-1 dose level refers to the dosing ladder. For example, 80 mg, 160 mg, 320 mg and 640 mg are the test doses, and determined RP2D is 320 mg. And then, RP2D-1 would be one dose level below that or 160 mg, and RP2D-2 would be 80 mg.

Upon completion of the DLT window (28 days) and depending on the number of evaluable patients and observed DLTs, the dose escalation and de-escalation for the combination of Compound 1 plus carfilzomib plus dexamethasone will be guided by the mTPI-2 method. After the completion of the dose-escalation/de-escalation process, an MTD for the combination of Compound 1 plus carfilzomib plus dexamethasone will be determined per the mTPI-2 method, and this MTD will be used together with other results, such as the totality of safety and preliminary efficacy to decide the final recommended dose for the combination of Compound 1 plus carfilzomib plus dexamethasone in Part 2 Cohorts 3 & 4.

### Part 2 (Cohort Expansion)

A cohort expansion is conducted to evaluate the safety and tolerability of Compound 1 at the RP2D as monotherapy and in combination with dexamethasone, the safety and tolerability of Compound 1 in combination with dexamethasone plus carfilzomib at the recommended dose for the combination therapy in patients with R/R MM and t (11;14). The study is also conducted to evaluate the efficacy of Compound 1 as monotherapy, in combination with dexamethasone, and with dexamethasone plus carfilzomib in patients with R/R MM and t (11;14) as measured by response or response rate.

Compound 1 monotherapy (Cohort 1): Patients are to receive Compound 1 monotherapy at the same dose level as the RP2D for Compound 1 in combination with dexamethasone. Patients whose disease progresses per International Myeloma Working Group (IMWG) criteria in the Compound 1monotherapy cohort (Cohort 1) may roll over into the combination therapy arm to receive Compound 1 plus dexamethasone and the highest dose of carfilzomib being tested in Part 2.

Compound 1 plus dexamethasone (Cohort 2): Patients are to receive Compound 1 in combination with dexamethasone (40 mg weekly), at the same dose level as the RP2D for Compound 1 in combination with dexamethasone.

Compound 1 plus dexamethasone and carfilzomib 70 mg/m2 weekly (Cohort 3): Patients will be enrolled in this cohort. This cohort may not open if the recommended dose for carfilzomib for the combination of Compound 1 plus carfilzomib is not determined at the carfilzomib dose of 70 mg/m2 per week.

Compound 1 plus dexamethasone and carfilzomib 56 mg/m2 weekly (Cohort 4): Patients will be enrolled in this cohort. If the dose combination of Compound 1 at RP2D-1 dose level plus carfilzomib at 56 mg/m2 per week+dexamethasone is tested and determined as not tolerable, there will be no expansion of cohorts (Cohorts 3 to 5) receiving Compound 1 plus dexamethasone plus carfilzomib in Part 2.

Compound 1 plus Carfilzomib 70 mg/m2 weekly plus dexamethasone (Cohort 5): Patients will be enrolled in this cohort. This cohort will serve as a control arm for Cohort 3 (and/or Cohort 4) in order to isolate the treatment effect and ensure safety of Compound 1. If there will be no expansion of cohorts receiving Compound 1 plus dexamethasone plus carfilzomib in Part 2, then Cohort 5 will not open either.

Carfilzomib dose is planned as 70 mg/m2 weekly when Cohort 3 opens in Part 2, but will be reduced to 56 mg/m2 weekly in the eventuality that Cohort 3 does not open due to intolerability.

### Main Inclusion Criteria:

1. Eastern Cooperative Oncology Group (ECOG) performance status of 0 to 2
2. A confirmed diagnosis of multiple myeloma (must have an M-component in serum and/or urine)
3. Measurable disease defined as:
   i. M-spike≥500 mg/dL, or ii. Urine protein M-spike of ≥200 mg/day, or iii. Serum free light chains≥10 mg/dL, and an abnormal K:λ ratio
4. Participant has documented relapsed or progressive MM on or after any regimen or who are refractory to the most recent line of therapy.
   i. Relapsed MM is defined as previously treated MM that progresses and requires initiation of salvage therapy but does not meet the criteria for refractory MM.
   ii. Refractory MM is defined as disease that is nonresponsive (failure to achieve minimal response or development of progressive disease) while on primary or salvage therapy or progresses within 60 days of last therapy.
      a. Participants in Part 1 should have failed all other available options including having had ≥3 prior lines of therapy including a proteasome inhibitor, IMiD agent, and an anti-CD38 monoclonal antibody.
      b. Participants in Part 2 should have had and failed ≥1 but ≤7 prior lines of therapy and will have had prior treatment with both a proteasome inhibitor and an IMiD agent. Note: A line of therapy consists of greater ≥1 complete cycle of a single agent, a regimen consisting of combination of several drugs, or a planned sequential therapy of various regimens. Induction therapy with consolidation and maintenance following stem cell transplant is considered a single line of therapy.
      c. Prior treatment with carfilzomib is allowed but the patient must not be considered carfilzomib refractory and not have had carfilzomib within the past 6 months
5. Positivity for t (11;14) by validated fluorescence in situ hybridization (FISH) testing assay in a pre-defined laboratory
   a. fresh bone marrow aspirate sample must be collected at screening and sent to central laboratory for t (11;14) FISH testing.
6. Adequate organ function defined as:
   a. Hemoglobin ≥8.0 g/dL, within 7 days before first dose of study treatment, independent of growth factor support and transfusions
   b. Platelet count ≥75,000/μL, within 7 days before first dose of study treatment, independent of growth factor support and transfusions
   c. Absolute neutrophil count (ANC)≥1000/mm3 [ANC=(% of segmented neutrophils + % of segmented bands)×total WBC count within 7 days before first dose of study treatment
   d. ALT and AST ≤3×upper limit of normal (ULN) and total bilirubin≤2.0×ULN Serum creatinine≤1.5×ULN or creatinine clearance≥45 mL/min/1.73 m2 calculated by the MDRD-6 formula.

### Main Exclusion Criteria:

1. Participant has any of the following conditions:
   a. Non secretory MM (Serum free light chains<10 mg/dL)
   b. Solitary plasmacytoma
   c. Active plasma cell leukemia (ie, either 20% of peripheral white blood cells or >2.0×109/L circulating plasma cells by standard differential)
   d. Waldenström macroglobulinemia
   e. Amyloidosis.
   f. Polyneuropathy, organomegaly, endocrinopathy, monoclonal protein, skin changes (POEMS) syndrome
   g. Uncontrolled diabetes (HbAlc>7% or 53 mmol/mol or requiring insulin at study entry
   h. Chronic respiratory disease that requires continuous oxygen
2. Significant cardiovascular disease, including but not limited to:
   a. Myocardial infarction≤6 months before screening
   b. Ejection fraction≤50%
   c. Unstable angina≤3 months before screening
   d. New York Heart Association Class III or IV congestive heart failure
   e. History of clinically significant arrhythmias (eg, sustained ventricular tachycardia, ventricular fibrillation, or torsades de pointes)
   f. Heart rate-corrected QT interval>480 milliseconds based on Fridericia's formula
   g. History of Mobitz II second-degree or third-degree heart block without a permanent pacemaker in place
   h. Uncontrolled hypertension at screening, defined as systolic blood pressure>170 mmHg and diastolic blood pressure>105 mmHg by ≥2 consecutive measurements
3. Known infection with human immunodeficiency virus (HIV)
4. Serologic status reflecting active viral hepatitis B (HBV) or viral hepatitis C (HCV) infection as follows:
   a. Presence of hepatitis B surface antigen (HBsAg) or hepatitis B core antibody (HBcAb). Participants with presence of HBcAb, but absence of HBsAg, are eligible if HBV DNA is undetectable (limitation of sensitivity<20 IU/mL),), and if they are willing to undergo monthly monitoring for HBV reactivation.
   b. Presence of HCV antibody. Participants with presence of HCV antibody are eligible if HCV RNA is undetectable (limitation of sensitivity<15 IU/mL).

### 2. Conclusions

In the combo treatment of Compound 1 and dexamethasone study, Compound 1 plus

dexamethasone was generally well tolerated in patients with R/R MM harboring t (11;14) at doses up to 640 mg, and initial safety and efficacy results were promising. The SMC has recommended 640 mg in combination with dexamethasone as the RP2D.

The PK of Compound 1 in combination with dexamethasone is available from MM patients who received the 80 to 640 mg target doses in this study. The available PK data showed that the steady state exposure (Cmax and AUC) of Compound 1 in combination with dexamethasone were comparable to that of Compound 1 monotherapy, indicating that the drug-drug-interaction (DDI) potential between dexamethasone and Compound 1 is low.

In this study, no patient experienced a treatment-emergent adverse event that led to treatment modification. No dose limiting toxicity (DLT) were seen across the 4 dose levels tested. No TEAEs lead to discontinuation. The most common TEAEs were insomnia (42%), fatigue (32%), nausea (26%), arthralgia (21%), COVID-19 (16%). Toxicities were rare and manageable. The only hematologic toxicity seen was 1 case of grade 2 neutropenia, which did not lead to dose modifications or discontinuation. These results suggested that Compound 1 in combination with dexamethasone is safe. Compound 1 in combination with dexamethasone appeared to be well tolerated in patients with R/R MM harboring t (11;14) at the dose levels tested in 4 dose escalation cohorts (80, 160, 320, and 640 mg once daily[QD]). In addition, based on preliminary efficacy data for 19 patients (3 patients at 80 mg, 3 patients at 160 mg, 3 patients at 320 mg, and 10 patients at 640 mg) in the 4 dose escalation cohorts, Compound 1 demonstrated activity at tested dose levels, and most patients achieved disease control. At 640 mg, ORR is 70%, VGPR or better is 40% (The results were shown in FIG. 4). In competitor data, ODD of venetoclax monotherapy in t (11; 14) MM was 40% with 27% of patients achieving VGPR.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

1. A method of treating multiple myeloma in a subject, the method comprising administering to the subject a therapeutically effective amount of a Bcl-2 inhibitor, wherein the Bcl-2 inhibitor is selected from the group consist of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2- isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)- 7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis (methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1- yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl (oxo)-16-sulfaneylidene)amino)cyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl (3-nitro-4-(((tetrahydro-2H-pyran-4-yl)methyl)amino)phenyl)(oxo)-16-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2- isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2- cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2- phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

2. A method of treating multiple myeloma in a subject, the method comprising administering to the subject a therapeutically effective amount of a Bcl-2 inhibitor, in combination with a therapeutically effective amount of dexamethasone, wherein the Bcl-2 inhibitor is selected from the group consist of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2- isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)- 7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis (methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1- yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl (oxo)-16- 2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl (3-nitro-4-(((tetrahydro-2H-pyran-4-yl)methyl)amino)phenyl)(oxo)-16-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2- isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2- cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2- phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

3. The mothed of claim 1 or 2, wherein the Bcl-2 inhibitor is 2-((1H-pyrrolo[2,3-b]pyridin-5-yl)oxy)-N-((4-((((1r,4r)-4-hydroxy-4-methylcyclohexyl)methyl)amino)- 3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof.

4. The method of claim 1 or 2, wherein the multiple myeloma is relapsed/refractory multiple myeloma.

5. The method of claim 1 or 2, wherein the multiple myeloma demonstrates cytogenetic abnormalities and/or gene expression changes.

6. The method of claim 1 or 2, wherein the multiple myeloma is t (11;14) positive multiple myeloma.

7. The method of claim 1 or 2, wherein the Bcl-2 inhibitor is orally administrated.

8. The method of claim 2, wherein the Bcl-2 inhibitor is administrated in combination with dexamethasone daily on a 21-day cycle.

9. The method of any one of claims 1-3, wherein the Bcl-2 inhibitor is orally administrated at a dose of 40 to 640 mg once daily.

10. The method of claim 1 or 2, wherein the Bcl-2 inhibitor is administrated at a dose of 40 mg once daily, 80 mg once daily, 160 mg once daily, 320 mg once daily, or 640 mg once daily.

11. The method of claim 2, wherein dexamethasone is administrated at a dose of 40 mg/m² once weekly.

12. The method of claim 9, wherein dexamethasone is administrated simultaneously with the Bcl-2 inhibitor.
